SEQUENCE LISTING (24 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 134 aa, Homo sapiens
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - DNA, 1413 bp, Homo sapiens
atggaatggagcggagtctttatctttctcctgtcagtaactgcaggtgttcactcccaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatggaggggggatggttactacgcgtacttcgatgtctggggcgcagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 4 - DNA, 717 bp, Homo sapiens
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 5 - protein, 470 aa, Homo sapiens
MEWSGVFIFLLSVTAGVHSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 238 aa, Homo sapiens
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - protein, 5 aa, Homo sapiens
NYLIE

SEQ ID NO: 8 - protein, 17 aa, Homo sapiens
VINPGSGGTNYNEKFKG

SEQ ID NO: 9 - protein, 12 aa, Homo sapiens
WRGDGYYAYFDV

SEQ ID NO: 10 - protein, 15 aa, Homo sapiens
KASQSVDYDGDSYMN

SEQ ID NO: 11 - protein, 7 aa, Homo sapiens
AASNLES

SEQ ID NO: 12 - protein, 9 aa, Homo sapiens
QQSNEDPYT

SEQ ID NO: 13 - DNA, 15 bp, Homo sapiens
aattacttgatagag

SEQ ID NO: 14 - DNA, 51 bp, Homo sapiens
gtgattaatcctggaagtggtggtactaactacaatgagaagttcaagggc

SEQ ID NO: 15 - DNA, 36 bp, Homo sapiens
tggaggggggatggttactacgcgtacttcgatgtc

SEQ ID NO: 16 - DNA, 45 bp, Homo sapiens
aaggccagccaaagtgttgattatgatggtgatagttatatgaac

SEQ ID NO: 17 - DNA, 21 bp, Homo sapiens
gctgcatccaatctagaatct

SEQ ID NO: 18 - DNA, 27 bp, Homo sapiens
cagcaaagtaatgaggatccgtacacg

SEQ ID NO: 19 - protein, 121 aa, Homo sapiens [sequence omitted]

SEQ ID NO: 20 - protein, 111 aa, Homo sapiens
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 21 - DNA, 363 bp, Homo sapiens
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatggaggggggatggttactacgcgtacttcgatgtctggggcgcagggaccacggtcaccgtctcctca

SEQ ID NO: 22 - DNA, 333 bp, Homo sapiens
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaa

SEQ ID NO: 23 - protein, 620 aa, artificial
  IL-2-(GxS)y-heavy chain; C-terminus of hIL-2 fused to N-terminus of variable heavy chain of NARA1
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 24 - protein, 388 aa, artificial
  IL-2-(GxS)y-light chain ; C-terminus of hIL-2 fused to N-terminus of variable light chain of NARA1
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2019/0016796 A1
(43) Pub. Date: Jan. 17, 2019

(54) COMBINATION THERAPY COMPRISING A SUPERAGONISTIC ANTIBODY AGAINST INTERLEUKIN-2 AND A CHECKPOINT BLOCKADE AGENT

(71) Applicant: UNIVERSITAT ZURICH, Zurich (CH)

(72) Inventors: Onur BOYMAN, Kusnacht (CH); Natalia ARENAS-RAMIREZ, Zurich (CH)

(73) Assignee: UNIVERSITAT ZURICH, Zurich (CH)

(21) Appl. No.: 16/068,694

(22) PCT Filed: Jan. 11, 2017
(86) PCT No.: PCT/EP2017/050477

§ 371 Date: Jul. 9, 2018

(30) Foreign Application Priority Data
Jan. 11, 2016 (EP) 16150708.2
Jul. 12, 2016 (EP) 16179132.2

(51) Int. Cl. C07K 16/24; C07K 16/28; A61P 35/00; C07K 14/55

(52) U.S. Cl. CPC C07K 16/246; C07K 16/2818; A61K 2039/507; C07K 14/55; C07K 2317/92; A61P 35/00

(57) ABSTRACT

The invention relates to a combination medicament comprising a human interleukin-2 (hIL-2)-specific monoclonal antibody (mAb), or antigen binding fragment thereof, the binding of which to hIL-2 inhibits binding of hIL-2 to CD25, and an immune checkpoint inhibitor agent. The hIL-2 antibody can be given without or with recombinant hIL-2 and is characterized by any of the parameters: the variable chain of the mAb comprises the amino acid sequence of SEQ ID NO 005 or SEQ ID NO 006; the binding to hIL-2 is characterized by a dissociation constant (K_{D})≤7.5 nmol/L; the binding to hIL-2 is characterized by an off-rate (K_{off})≤1×10⁻⁴ s⁻¹ and/or the antibody displays no measurable cross-reactivity to murine IL-2.

Malignant melanoma is a frequent cancer type. The 5-year survival rate of metastatic melanoma is about 15%, which currently available treatment strategies barely improve.

Interleukin-2 (IL-2) is a cytokine able to potently stimulate cytotoxic lymphocytes against metastatic tumours. IL-2 however also stimulates so-called CD25⁺ CD4⁺ regulatory T cells (Treg cells) that are crucial for prevention of autoimmune disease. Treg cells can significantly dampen anti-tumour responses by cytotoxic lymphocytes, thus antagonizing the beneficial anti-tumour effects of IL-2. IL-2 can exert toxic adverse effects at doses required to achieve a clinical anti-tumour response.

Standard IL-2 immunotherapy has been used for the immunotherapy of metastatic melanoma and metastatic renal cell carcinoma. While IL-2 given at high doses has shown objective response rates in about 17% and complete regression in about 6-9% of patients, IL-2 given at these doses frequently led to toxic adverse effects.

Previous work by the inventors has provided a human interleukin-2 (hIL-2)-specific monoclonal antibody (mAb) that inhibits binding of hIL-2 to CD25 and potently stimulates cytotoxic cells, but not Treg cells. In subsequent work, the inventors tried to elucidate the potential of this antibody to be further improved by combination with immune modulatory approaches.

The problem underlying the present invention is to improve the existing therapy based on anti-human IL-2 monoclonal antibodies able to recognize and bind a specific epitope of human IL-2, thereby enabling stimulation of cytotoxic T cells, but not of Treg cells. This problem is solved by the subject-matter of the independent claims.

### Terms and Definitions

Identity in the context of the present specification is a single quantitative parameter representing the result of a sequence comparison position by position. Methods of sequence comparison are known in the art; the BLAST algorithm available publicly is an example. One example for comparison of amino acid sequences is the BLASTP algorithm that uses default settings such as: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear

In the context of the present specification, the term antibody is used in its meaning known in the art of cell biology and immunology; it refers to whole antibodies, any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxyterminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system.

In the context of the present specification, the term antigen binding portion or antigen binding fragment is used in its meaning known in the art of cell biology and immunology; it refers to one or more fragments of an intact antibody that retain the ability to specifically bind to a given antigen (e.g., interleukin-2). Antigen binding functions of an antibody can be performed by fragments of an intact antibody. Examples of binding fragments encompassed within the term antigen binding portion or antigen binding fragment of an antibody include a Fab fragment, a monovalent fragment consisting of the V_{L}, V_{H}, C_{L} and C_{H} domains; a F(ab)₂ fragment, a bivalent fragment comprising two Fab fragments linked by a disulfide bridge at the hinge region; an Fd fragment consisting of the V_{H} and C_{H} domains; an Fv fragment consisting of the V_{L} and V_{H} domains of a single arm of an antibody; a single domain antibody (dAb) fragment, which consists of a V_{H} domain or a V_{L} domain; and an isolated complementarity determining region (CDR).

In the context of the present specification, the term chimeric antibody is used in its meaning known in the art of cell biology and immunology; it refers to an antibody molecule in which the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, cytokine, toxin, hormone, growth factor, drug, etc. For example, an antibody can be modified by replacing its constant region with a cytokine. Due to the replacement with a cytokine, the chimeric antibody can retain its specificity in recognizing the antigen while having also the function, or part thereof, of the original cytokine molecule.

In the context of the present specification, the term hybridoma is used in its meaning known in the art of cell biology and biochemistry; it refers to a hybrid cell created by fusion of a specific antibody-producing B-cell with a myeloma (B-cell cancer) cell. Hybridoma cells can be grown in tissue culture and produce antibodies of a single specificity (monoclonal antibodies).

In the context of the present specification, the term single-chain variable fragment (scFv) is used in its meaning known in the art of cell biology and biochemistry; it refers to a fusion protein of the variable regions of the heavy (V_{H}) and light chains (V_{L}) of immunoglobulins, connected with a short linker peptide of ten to about 25 amino acids. The scFv retains the specificity of the original immunoglobulin, despite removal of the constant regions and the introduction of the linker.

In the context of the present specification, the term fragment antigen-binding (Fab) is used in its meaning known in the art of cell biology and immunology; it refers to a region on an antibody that binds to antigens. It is composed of one constant and one variable domain of each of the heavy (V_{H}) and light chains (V_{L}) of immunoglobulins. These domains shape the antigen-binding site at the amino terminal end of the monomer.

In the context of the present specification, the term dissociation constant (K_{D}) is used in its meaning known in the art of chemistry and physics; it refers to an equilibrium constant that measures the propensity of a larger object to dissociate reversibly into smaller components, as when a complex falls apart into its component molecules. K_{D} is expressed in molar units [M] and corresponds to the concentration of [Ab] at which the binding sites of [Ag] are half occupied. In other words the concentration of unbound [Ab] equals the concentration of the [AbAg] complex. The dissociation constant can be calculated according to the following formula:

$${K}_{D} = \frac{[ Ab ] * [ Ag ]}{[ AbAg ]}$$

[Ab]: concentration of antibody; [Ag]: concentration of antigen; [AbAg]: concentration of antibody-antigen complex

In the context of the present specification, the terms off-rate (K_{off}; [1/sec]) and on-rate (Kₒₙ; [1/sec*M]) are used in their meaning known in the art of chemistry and physics; they refer to a rate constant that measures the dissociation (K_{off}) or association (Kₒₙ) of an antibody with its target antigen. K_{off} and Kₒₙ can be experimentally determined using methods well established in the art. A method for determining the K_{off} and Kₒₙ of an antibody employs surface plasmon resonance. This is the principle behind biosensor systems such as the Biacore® or the ProteOn® system. They can also be used to determine the dissociation constant K_{D} by using the following formula:

$${K}_{D} = \frac{{K}_{off}}{{K}_{on}}$$

In the context of the present specification, the term humanized antibodies is used in its meaning known in the art of cell biology and biochemistry; it refers to antibodies originally produced by immune cells of a non-human species, whose protein sequences have been modified to increase their similarity to antibody variants produced naturally in humans.

In the context of the present specification, the term no measurable cross-reactivity refers to the lacking capability of an antibody to recognize and bind to orthologous proteins from other species. For example, an antibody directed against human interleukin-2 would have no measurable cross-reactivity to murine interleukin-2 if, under suitable conditions, binding of the antibody to murine interleukin-2 could not be detected with sufficiently sensitive methods such as surface plasmon resonance. One such example of no measurable cross-reactivity is shown in FIG. 9 for the antibody in the lower panel (NARA1).

In the context of the present specification, the term “human interleukin-2” or “hIL-2” refers to the protein designated UniProt ID P60568 (SEQ ID NO 001).

According to a first aspect of the invention, a combination medicament is provided, wherein the combination medicament comprises:
a human interleukin-2 (hIL-2)-specific monoclonal antibody (mAb), or antigen binding fragment thereof, wherein the antibody is able to bind to a particular epitope in hIL-2 and thereby inhibits binding of hIL-2 to CD25, thus abrogating the immunological effects of interaction of hIL-2 with CD25 (particularly Treg stimulation), and
an immune checkpoint inhibitor agent selected from an inhibitor of cytotoxic T-lymphocyte-associated protein 4 (CTLA-4; also known as CD152) interaction with CD80 or CD86, an inhibitor of the interaction of programmed cell death protein 1 (PD-1; also known as CD279) with its ligand PD-L1, a ligand of T cell immunoglobulin and mucin domain-containing 3 (TIM-3), an inhibitor of the interaction of B lymphocyte and T lymphocyte attenuator (BTLA) with herpes virus entry-mediator (HVEM, also known as TNFRSF14), and an inhibitor of the interaction of lymphocyte activation gene 3 protein (LAG3) with galectin 3.

The human interleukin-2 (hIL-2)-specific monoclonal antibody, or antigen binding fragment thereof, is further characterized by at least one of the parameters:
a) the variable chain of the mAb comprises an amino acid sequence having an identity of ≥85%, ≥90%, ≥95%, or ≥99% compared to SEQ ID NO 005 or SEQ ID NO 006; and/or
b) the antibody binding to hIL-2, i.e. the reaction mAb+hIL-2⇔mAb*hIL-2, wherein mAb*hIL-2 symbolizes the bound complex of antibody and interleukin, is characterized by a dissociation constant (K_{D})≤7.5 nmol/L, ≤5 nmol/L, ≤3 nmol/L, ≤2 nmol/L or ≤1.5 nmol/L; and/or
c) the antibody binding to hIL-2 is characterized by an off-rate (K_{off})≤1×10⁻⁴ s⁻¹, ≤8×10⁻⁵ s⁻¹, ≤6×10⁻⁵ s⁻¹, ≤4×10⁻⁵ s⁻¹, ≤3×10⁻⁵ s⁻¹ or ≤2.1×10⁻⁵ s⁻¹;
d) upon mAb binding to hIL-2, the resulting mAb*hIL-2 complex cannot efficiently bind human IL-2 receptor α (also known as CD25) anymore, effectively rendering the binding of human CD25 to mAb*hIL-2 to background levels as compared to the binding of human CD25 to free (non-complexed) hIL-2 when measured by surface plasmon resonance; and/or
e) the antibody or antigen binding fragment thereof binds to a specific human interleukin-2 (hIL-2) epitope which comprises the amino acids K52, P54, K55, T57, R58, T61, F62, K63, Q94, and K96 of hIL-2; and/or
f) the antibody displays no measurable cross-reactivity to murine IL-2.

A lack of cross-reactivity with murine IL-2 is advantageous for preclinical studies, which usually involve mouse models, such as the use of mAb*hIL-2 complexes for the treatment of murine tumour models where a cross-reactive anti-IL-2 mAb might bind and seclude endogenous murine IL-2 from endogenous murine Treg cells, thus enhancing the anti-tumour response.

A lack of cross-reactivity with murine IL-2 is also advantageous for preclinical safety and efficacy studies conducted prior to development of a candidate mAb in human patients.

In certain embodiments, the hIL-2 mAb comprises at least one V_{H} and/or V_{L} sequence having an identity of ≥80%, ≥85%, ≥90%, ≥92%, ≥93%, ≥94%, ≥95%, ≥96%, ≥97% or ≥98% compared to SEQ ID NOs 019 or SEQ ID NO 020.

In certain embodiments, the variable chain of the hIL-2 mAb comprises an amino acid sequence having an identity of ≥85%, ≥90%, ≥95%, or ≥99% compared to SEQ ID NOs 003, 004, 005 or 006 and the hIL-2 mAb is characterized by a dissociation constant ≤7.5 nmol/L, ≤5 nmol/L, ≤3 nmol/L, ≤2 nmol/L or ≤1.5 nmol/L.

In certain embodiments, the variable chain of the hIL-2 mAb comprises an amino acid sequence having an identity of ≥85%, ≥90%, ≥92%, ≥93%, ≥94%, ≥95%, ≥96%, ≥97%, ≥98% or ≥99% compared to SEQ ID NO 005 or 006 and the hIL-2 mAb is characterized by an off-rate ≤1×10⁻⁴ s⁻¹, ≤8×10⁻⁵ s⁻¹, ≤6×10⁻⁵ s⁻¹, ≤4×10⁻⁵ s⁻¹, ≤3×10⁻⁵ s⁻¹ or ≤2.1×10⁻⁵ s⁻¹.

In certain embodiments, the variable chain of the hIL-2 mAb comprises an amino acid sequence having an identity of ≥85%, ≥90%, ≥92%, ≥93%, ≥94%, ≥95%, ≥96%, ≥97%, ≥98% or ≥99% compared to SEQ ID NO 005 or 006 and the hIL-2 mAb displays no measurable cross-reactivity to murine IL-2.

In certain embodiments, the hIL-2 mAb or antigen binding fragment thereof binds to a human interleukin-2 (hIL-2) epitope which comprises the amino acids K52, P54, K55, T57, R58, T61, F62, K63, Q94, and K96, and which comprises any one or more of the amino acids N50, N53, N91, L92, A93, and N97.

In certain embodiments, the hIL-2 mAb or antigen binding fragment thereof comprises an antigen recognition surface having epitope recognition characteristics equivalent to an antibody or antigen binding fragment to a specific human interleukin-2 (hIL-2) epitope which comprises the amino acids K52, P54, K55, T57, R58, T61, F62, K63, Q94, and K96 of hIL-2.

In certain embodiments, the hIL-2 mAb or antigen binding fragment thereof comprises an antigen recognition surface having epitope recognition characteristics equivalent to an antibody or antigen binding fragment to a specific human interleukin-2 (hIL-2) epitope which comprises the amino acids K52, P54, K55, T57, R58, T61, F62, K63, Q94, and K96 of hIL-2 and which comprises any one or more of the amino acids N50, N53, N91, L92, A93, and N97.

In certain embodiments, the sequence of the hIL-2 mAb is humanized for administration to human patients to prevent adverse reactions.

In certain embodiments, the hIL-2 mAb is provided as fragment antigen-binding (Fab) or single-chain variable fragment (scFv).

In certain embodiments, the hIL-2 mAb comprises at least one complementarity determining (CDR) sequence having an identity of ≥80%, ≥85%, ≥90%, ≥92%, ≥93%, ≥94%, ≥95%, ≥96%, ≥97% or ≥98% compared to SEQ ID NOs 007, 008, 009, 010, 011 or 012.

In certain embodiments, the hIL-2 mAb comprises at least three different complementarity determining (CDR) sequences, each of which is ≥80%, ≥85%, ≥90%, ≥92%, ≥93%, ≥94%, ≥95%, ≥96%, ≥97% or ≥98% or even 100% identical to one of SEQ ID NO 007, SEQ ID NO 008, SEQ ID NO 009, SEQ ID NO 010, SEQ ID NO 011 or SEQ ID NO 012.

In certain embodiments, the hIL-2 mAb comprises at least four, five or six different complementarity determining (CDR) sequences, each of which is ≥80%, ≥85%, ≥90%, ≥92%, ≥93%, ≥94%, ≥95%, ≥96%, ≥97% or ≥98% or even 100% identical to one of SEQ ID NO 007, SEQ ID NO 008, SEQ ID NO 009, SEQ ID NO 010, SEQ ID NO 011 or SEQ ID NO 012, respectively.

In certain embodiments, the sequence of the hIL-2 mAb is encoded by a nucleic acid molecule having ≥60%, ≥70%, ≥80%, ≥85%, ≥90%, ≥92%, ≥93%, ≥94%, ≥95%, ≥96%, ≥97%, ≥98% or ≥99% sequence identity compared to SEQ ID NOs 003 and/or 004.

The skilled person is aware that an antibody molecule is usually composed of two separate amino acid chains, which in turn on the level of mRNA are encoded by two separate nucleic acid molecules, namely one encoding the heavy chain (with constant and variable regions) and one encoding the light chain (with constant and variable regions). Transgene expression of such two amino acid chains encoding the light and heavy chain will commonly be effected from one transgene expression construct (the nucleic acid molecule). The skilled person however will also be able to find a way to express the two amino acid chains constituting the antibody of the present invention from two different nucleic acid molecules, or to join the two amino acid chains by a linker. In the context of the present specification, the expression “the sequence of the hIL-2 mAb is encoded by a nucleic acid molecule that has ≥98% sequence identity compared to SEQ ID NOs 003 (the heavy chain encoding sequence) and 004 (the light chain encoding sequence)” is synonymous to “the sequence of the hIL-2 mAb is encoded by one or two (separate) nucleic acid molecules encoding one or two (separate) amino acid chains that comprise the sequence encoded by SEQ ID NO 3 and the sequence encoded by SEQ ID NO 4, from which the antibody is constituted”.

In certain embodiments, the sequence of the hIL-2 mAb is encoded by a (at least one, in certain embodiments two) nucleic acid molecule(s) comprising one, two, three, four, five or six sequence tracts characterized by a sequence identity value ≥90%, ≥92%, ≥93%, ≥94%, ≥95%, ≥96%, ≥97%, ≥98% or ≥99% when compared to a sequence selected from one, two, three, four, five or six sequences of the group SEQ ID NO 013, SEQ ID NO 014, SEQ ID NO 015, SEQ ID NO 016, SEQ ID NO 017 and SEQ ID NO 018. The skilled person is aware that in instances where two, three, four, five or six sequence tracts are comprised in the sequence of the hIL-2 mAb, these sequences may encode CDR sequences comprised on different parts of the antibody amino acid sequence (i.e. the heavy and light chain, respectively).

In certain embodiments, the sequence of the hIL-2 mAb is encoded by a (at least one, in certain embodiments two) nucleic acid molecule(s) having ≥60%, ≥70%, ≥80%, particularly ≥85%, ≥90%, ≥92%, ≥93%, ≥94%, ≥95%, ≥96%, ≥97%, ≥98% or ≥99% sequence identity compared to SEQ ID NOs 021 and/or 022.

In certain embodiments, the combination medicament further comprises human interleukin-2.

According to an alternative aspect of the invention, a combination medicament is provided that contains an IL-2/IL-2mAB component and a checkpoint inhibitor. The IL-2/IL-2mAB component provides the stimulatory effect of IL-2, concomitantly blocking the signals of IL-2 that provide the effect on Treg cells.

In certain embodiments, the combination medicament further comprises human IL-2.

In certain embodiments, the combination medicament comprises
a. a fusion protein comprising:
   i. a human interleukin-2 (hIL-2) specific binding polypeptide fragment, wherein said polypeptide fragment is characterized by any one of the parameters:
      binding of said polypeptide fragment to hIL-2 inhibits binding of hIL-2 to CD25, and/or
      the hIL-2 binding polypeptide fragment comprises an amino acid sequence having an identity of ≥85%, ≥90%, ≥92%, ≥93%, ≥94%, ≥95%, ≥96%, ≥97% or ≥98% compared to SEQ ID NO 019 or SEQ ID NO 020, and/or
      the binding to hIL-2 is characterized by a dissociation constant (K_{D}) ≤7.5 nmol/L, ≤5 nmol/L, ≤3 nmol/L, ≤2 nmol/L or ≤1.5 nmol/L; and/or
      the binding to hIL-2 is characterized by an off-rate (K_{off}) 1×10⁻⁴ s⁻¹, ≤8×10⁻⁵ s⁻¹, ≤6×10⁻⁵ s⁻¹, ≤4×10⁻⁵ s⁻¹, ≤3×10⁻⁵ s⁻¹ or ≤2.1×10⁻⁵ s⁻¹; and/or
      the antibody displays no measurable cross-reactivity to murine IL-2;
   ii. a human IL-2 polypeptide fragment characterized by the biological activity of IL-2, particularly characterized by the ability to stimulate CD8+ T cells, wherein the IL-2 polypeptide has an identity of ≥85%, ≥90%, ≥92%, ≥93%, ≥94%, ≥95%, ≥96%, ≥97% or ≥98% compared to SEQ ID NO 001, and, optionally,
   iii. an amino acid linker of 1 to 50, particularly of 5 to 40, more particularly of 10 to 30, even more particularly of approx. 15 to 25 amino acids, linking the hIL-2 binding polypeptide fragment to the human IL-2 polypeptide fragment as one single polypeptide chain; and
b. an immune checkpoint inhibitor selected from
   i. an inhibitor of CTLA-4 interaction with CD80 or CD86, particularly an antibody specific for CTLA-4;
   ii. an inhibitor of PD-1/PD-L1 interaction, particularly an antibody specific for PD-1 or PD-L1;
   iii. an inhibitor of TIM-3 interaction with its physiological partner, particularly a ligand of TIM-3, more particularly an antibody against TIM-3;
   iv. an inhibitor of the interaction of BTLA with HVEM; and
   v. an inhibitor of the interaction of LAG3 with galectin 3.

In other words, the fusion protein retains the ability of the antibody to bind and direct human interleukin-2 to stimulate selected immune cells, such as CD8⁺ T cells and NK cells. The IL-2 portion of the molecule will be essentially the sequence of IL-2, but the skilled person understands that small sequence changes might be tolerated that retain the biological activity of IL-2, particularly its ability to stimulate cytotoxic effector T-cells.

The advantage of using such fusion protein is that human IL-2 will not be able to dissociate from the antibody and that the therapy will be composed of one single product instead of two, facilitating various aspects of manufacture, dosing and regulatory compliance.

In certain embodiments of any of the aspects of the invention provided herein, the hIL-2 mAb or antigen binding fragment thereof binds to a human interleukin-2 (hIL-2) epitope which further comprises the amino acids N50, N53, N91, L92, A93, and N97 of hIL-2.

In certain embodiments of any of the aspects of the invention provided herein, the immune checkpoint inhibitor agent is an antibody specifically binding to CTLA-4, CD80, CD86, PD-1, PD-L1, TIM-3, BTLA, HVEM, LAG3 or galectin 3.

In certain embodiments of any of the aspects of the invention provided herein, the immune checkpoint inhibitor agent is a non-agonistic antibody specifically binding to CTLA-4, CD80, CD86, PD-1, PD-L1, TIM-3, BTLA, HVEM, LAG3 or galectin 3.

In certain embodiments of any of the aspects of the invention provided herein, the immune checkpoint inhibitor agent is an inhibitor of interaction of CTLA-4 with CD80 or CD86.

In certain embodiments of any of the aspects of the invention provided herein, the immune checkpoint inhibitor agent is ipilimumab (Yervoy; CAS No. 477202-00-9). In certain embodiments of any of the aspects of the invention provided herein, the immune checkpoint inhibitor agent is nivolumab (Opdivo; CAS No. 946414-94-4). In certain embodiments of any of the aspects of the invention provided herein, the immune checkpoint inhibitor agent is pembrolizumab (Keytruda; CAS No. 1374853-91-4). In certain embodiments of any of the aspects of the invention provided herein, the immune checkpoint inhibitor agent is atezolizumab (Tecentriq; CAS No. 1380723-44-3).

According to another aspect of the invention, the combination medicament according to any one of the previous aspects or embodiments is provided for use in therapy of cancer.

In certain embodiments of this aspect of the invention, the combination medicament is provided for use in therapy of malignant melanoma, particularly metastatic malignant melanoma. Both, IL-2 immunotherapy and checkpoint inhibitors, such as anti-PD-1/PD-L1 and anti-CTLA-4, have shown to be beneficial in the treatment of metastatic malignant melanoma.

In certain embodiments of this aspect of the invention, the combination medicament is provided for use in therapy of renal cell cancer. IL-2 immunotherapy has been shown to be beneficial in the treatment of renal cell cancer.

In certain embodiments of this aspect of the invention, the combination medicament is provided for use in therapy of lung cancer. In certain embodiments of this aspect of the invention, the combination medicament is provided for use in therapy of bladder cancer.

Lung cancer and bladder cancer have been shown to be responsive to treatment with immune checkpoint inhibitors that prevent PD-1/PD-L1 interaction.

In certain embodiments of this aspect of the invention, the combination medicament is provided for use in therapy of solid cancer with a regular to frequent load of somatic mutations, also termed cancer neoantigens, in particular melanoma, lung cancer, stomach cancer, esophagus cancer, colorectal cancer, bladder cancer, uterus cancer, cervix cancer, liver cancer, head and neck cancer, kidney cancer, breast cancer, and pancreas cancer. Such cancers have been shown to be responsive to treatment with immunotherapy.

In the context of the present specification, a “regular load of somatic mutations” is defined as 1-10 somatic mutations per megabase of coding DNA, corresponding to 15-150 nonsynonymous mutations within expressed genes, and a “frequent load of somatic mutations” is defined as 10-100 somatic mutations per megabase of coding DNA, corresponding to 150-1500 nonsynonymous mutations within expressed genes (Alexandrov et al., Nature. 2013 Aug. 22; 500(7463):415-21; Schumacher and Schreiber, Science. 2015 Apr. 3; 348(6230):69-74).

Wherever alternatives for single separable features such as, for example, a coding sequence or binding epitope are laid out herein as “embodiments”, it is to be understood that such alternatives may be combined freely to form discrete embodiments of the invention disclosed herein.

The invention is further illustrated by the following items, examples and figures, from which further embodiments and advantages can be drawn. These examples are meant to illustrate the invention but not to limit its scope.

### BRIEF DESCRIPTION OF THE FIGURES

FIG. 1 shows anti-human IL-2 binders. Supernatants of B cell clones obtained after B cell hybridoma fusion were added to a plate previously coated with human IL-2. The anti-human IL-2 mAbs were detected using a biotinylated anti-mouse IgG antibody.

FIG. 2 shows screening of anti-human IL-2 mAbs for binding to presumed specific human IL-2 epitope. Plates were coated with 5344 (a hIL-2 mAb without the herein targeted superagonistic behaviour) and blocked, followed by addition of human IL-2 in order to allow the cytokine to bind to 5344, thus covering a specific epitope of the IL-2. Then the supernatants giving a positive signal in the first screening (see FIG. 1) were added. After allowing the mAbs in the supernatants to bind to the IL-2-5344 complex, a biotinylated MAB602 antibody was added to the plate in order to assess whether the tested mAbs of the supernatants bound to the same (so-called “competitors”) or to a different region than MAB602. The competitor mAbs led to an absorbance (OD450) that is two-fold lower than the absorbance obtained with MAB602 alone (in this case OD=1.1, as shown in H11).

FIG. 3 shows concentration-dependent competition of B cell hybridomas. The supernatants of 8 competitor B cell hybridoma clones of the first screening (see FIG. 2) were expanded and concentrated before use in this assay. The supernatants of these 8 competitor B cell hybridoma clones (labeled 1 to 8) were added in increasing quantities. Competent competitor B cell hybridoma clones reduced the OD450 as much as MAB602 or even more, which is evident for clones 1 and 2. MAB602 at different concentrations (green open circles) served as a control.

FIG. 4 shows in vivo proliferation of CD8⁺ T cells. Carboxyfluorescein succinimidyl ester (CFSE)-labeled CD8⁺ T cells of CD45.1-congenic IL-7 transgenic mice were transferred to CD45.2-congenic WT recipient mice, followed by daily injections of phosphate-buffered saline (PBS), IL-2, IL-2 plus MAB602 (IL-2/MAB602), IL-2 plus 5344 (IL-2/5344), IL-2 plus hybridoma 1 (IL-2/Hyb#1), or IL-2 plus hybridoma 2 (IL-2/Hyb#2) for 4 days. On day 5, lymph nodes and spleens were analyzed for CFSE profiles of donor CD45.1⁺ CD8⁺ T cells. Shown are the results obtained with the lymph nodes, similar results were obtained in the spleens.

FIG. 5 shows phenotypic characterisation of endogenous CD8⁺ T cells and NK cells following in vivo treatment using IL-2 plus hybridoma 1 and 2. Mice were treated as in FIG. 4, followed by assessment by flow cytometry of endogenous CD8⁺ T cell subsets and NK cells in the lymph nodes and spleen. Shown are (A) CD8 vs. CD3 profiles of total lymph node cells (left graphs) and CD44 (activated or memory T cells) vs. CD122 (IL-2 receptor β-subunit, present on activated or memory T cells) profiles of CD3⁺ CD8⁺ lymph node cells, or (B) NK1.1 vs. CD3 profiles of mice receiving the indicated treatment. Activated/memory CD8⁺ T cells are high for CD44 and intermediate to high for CD122. NK cells are CD3 negative and NK1.1 positive. Similar results were obtained using spleen cells.

FIG. 6 shows total cell counts of activated/memory CD8⁺ T cells and NK cells in lymph nodes and spleens. Animals were treated and analyzed as in FIG. 5. Shown are absolute cell counts of CD44high CD8⁺ T cells (so-called memory-phenotype, MP CD8⁺ cells (red, lower bars)) and of CD3 negative NK1.1⁺ NK cells (black, upper bars) in lymph nodes (top panel) and spleen (lower panel).

FIG. 7 shows surface plasmon resonance binding curves of the commercially available monoclonal antibody MAB602 (A) and the monoclonal antibody NARA1 (B), which is an example of the present invention, to human IL-2. For this experiment an amine coupling GLM chip was used. The activation of the carboxylic acid groups in the chip was done using a mix of 1-ethyl-3-3-dimethylaminopropyl carbodiimide hydrochloride (EDC at 0.2 M) and sulfo N-hydroxysulfosuccinimides (s_NHS at 0.05M) at 30 μl/min for 420 seconds (s). The antibodies NARA1 and MAB602 were coated in the chip at 100 μg/ml in a sodium acetate buffer (10 mM pH 4.5). Deactivation was followed adding ethanolamine HCl at 30 μl/min for 300 s. Finally human IL-2 was added at different concentrations (starting from 100 nM and followed by three-fold dilutions) at 100 μl/min, 600 s association, and 240 s dissociation. The response is concentration dependent, with 100 nM concentrations (red line) giving the most pronounced response.

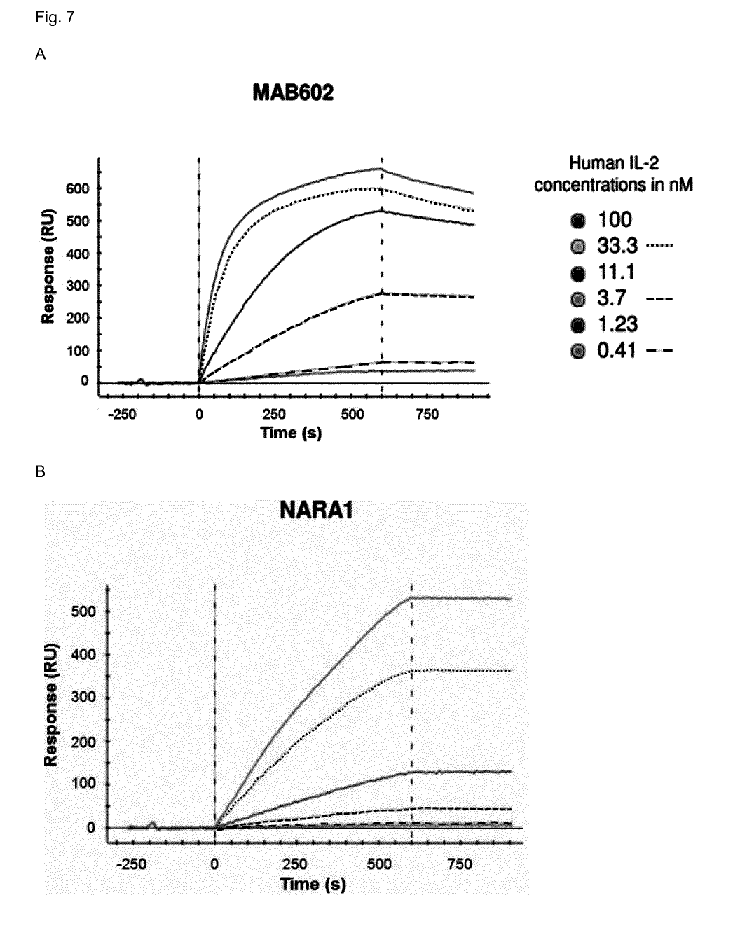

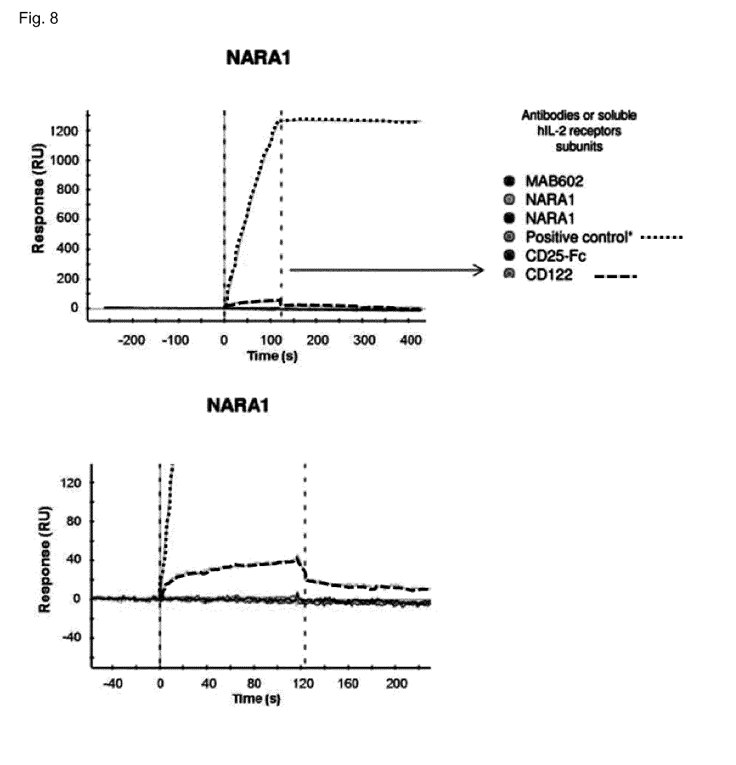

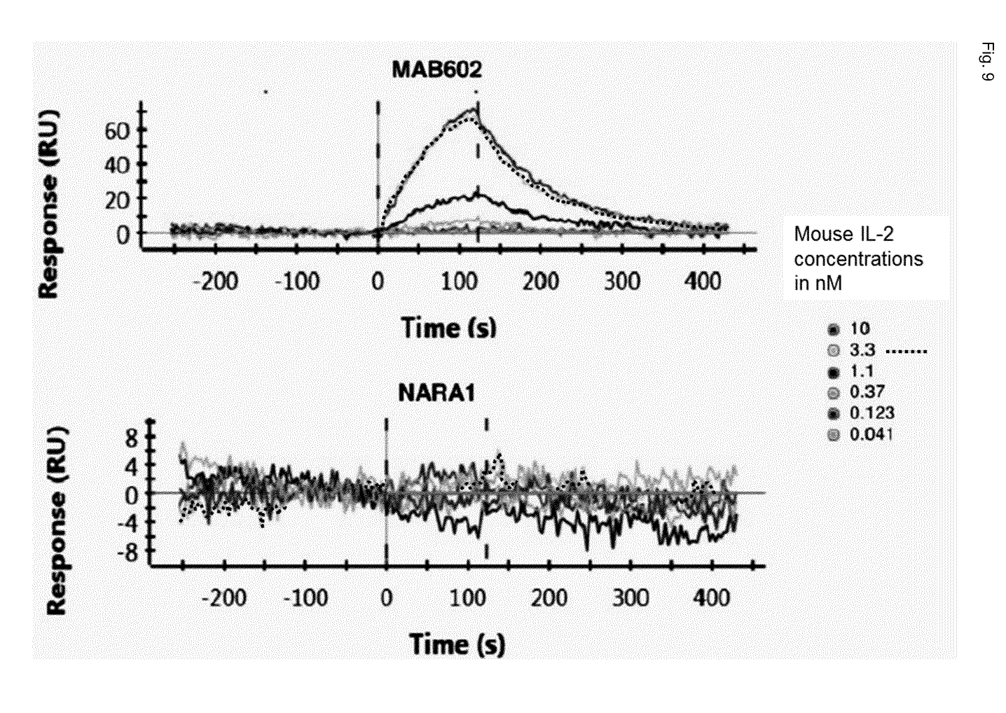

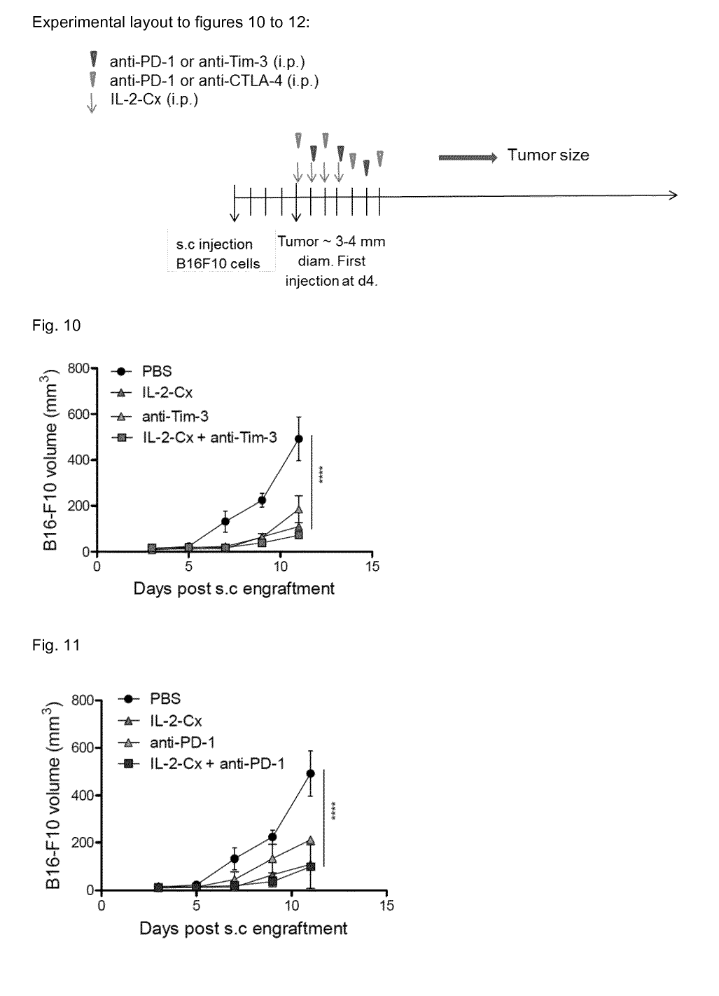

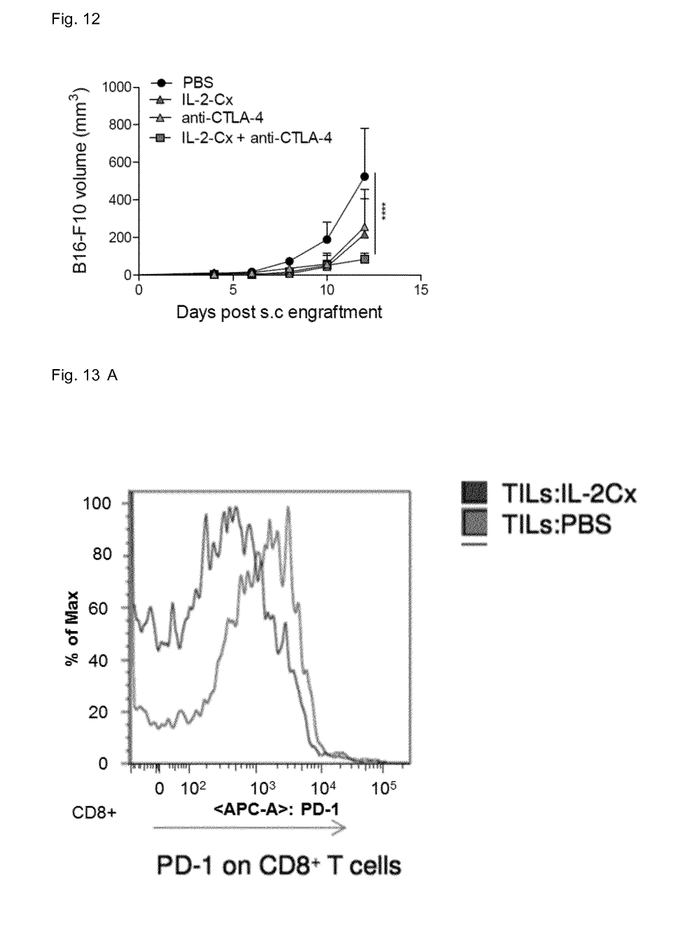

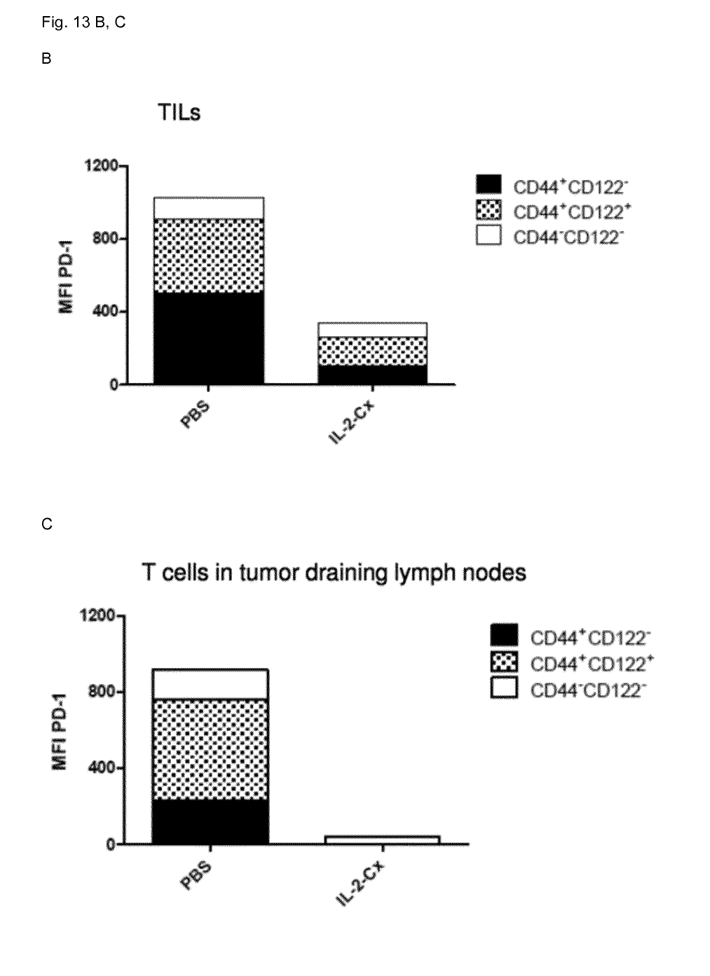

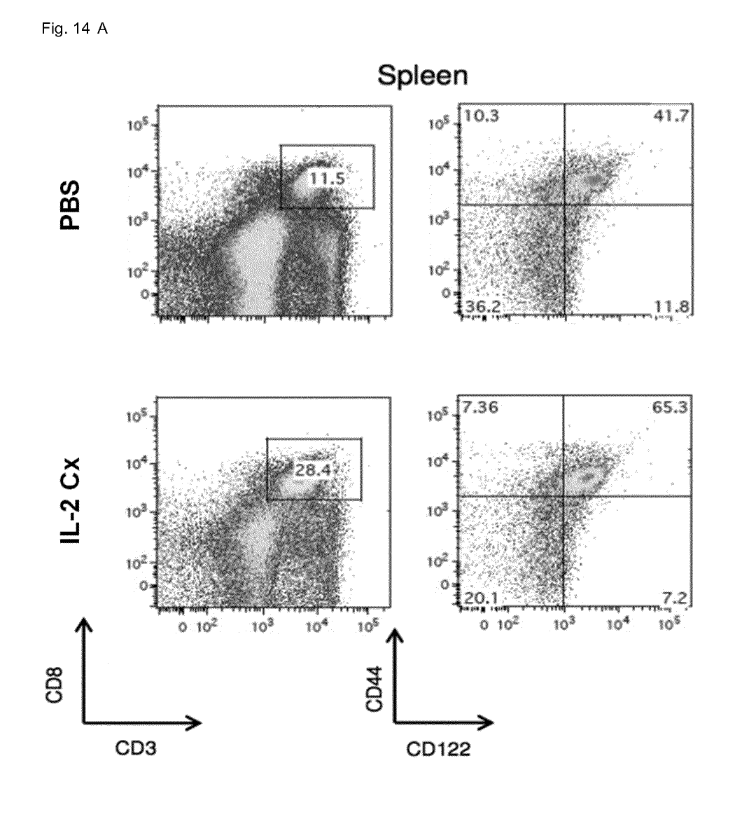

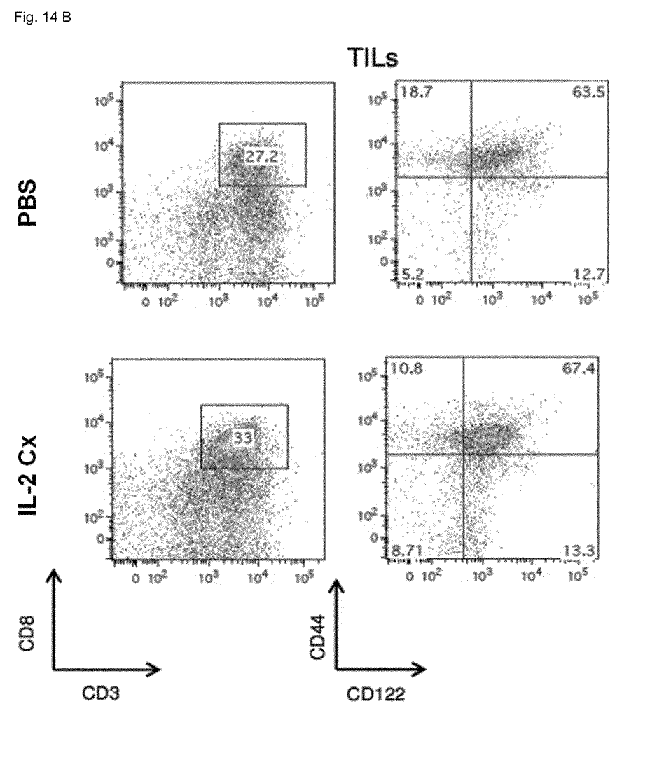

FIG. 8 shows surface plasmon resonance binding curves of human IL-2 bound to the monoclonal antibody NARA1 with the IL-2 receptors subunits CD25 (used here as an Fc fusion of CD25-Fc), CD122, the monoclonal antibody MAB602 or an anti-hIL-2 antibody binding to a different human IL-2 epitope than NARA1 and MAB602. The chip described in FIG. 7 coated with NARA1 and MAB602 was re-used. Regeneration of the chip was done using 10 mM glycine, pH 2.5, 30 μl/min, 60 s. Human IL-2 was added at saturating concentration (1 μM), at 100 μl/min, 120 s association, and 0 s dissociation. Immediately after IL-2 association to the antibodies, the second analytes were added at 100 μl/min, 120 s association, and 240s dissociation. The concentration used for the cross-binding were: MAB602: 50 nM; NARA1: 50 nM; positive control: 50 nM; CD25-Fc: 500 nM; CD122: 138 nM.
When hIL-2 is bound to NARA1, an anti-hIL-2 antibody that recognizes a different hIL-2 epitope (here termed ‘positive control’, green line)) binds strongly to the hIL-2/NARA1 complex as expected (green line in FIG. 8). Alternatively, IL-2Rα (in the form of CD25-Fc) cannot bind to hIL-2 when hIL-2 is already bound to NARA1 (pink line, FIG. 8), however, IL-2Rβ (CD122) still binds to hIL-2 when hIL-2 is already bound to NARA1 (orange line, FIG. 8).

FIG. 9 shows surface plasmon resonance binding curves of the monoclonal antibodies MAB602 (top graph) and NARA1 (lower graph) to murine IL-2. The same chip used for the generation of the data in FIGS. 7 and 8 was re-used. Regeneration of the chip was done with 10 mM glycine, pH 2.5, 30 μl/min, 60 s.
Mouse IL-2 (mIL-2) or human IL-2 (hIL-2) starting at 10 nM and then doing a three-fold dilution was injected at 100 μl/min, 120 s association, and 240 s dissociation.
In the top graph MAB602 shows cross-reactivity by binding to mouse IL-2. Especially, with higher concentrations of murine interleukin-2 (>1 nM) the binding curves differ significantly from background levels with response units (RU) well above 10. Whereas NARA1 (lower graph) displays no measurable cross-reactivity to murine IL-2 at all concentrations tested.

FIG. 10 shows anti-tumor effects in C57BL/6 mice harboring syngeneic subcutaneous B16F10 melanoma nodule following treatment with IL-2 complexes and/or anti-Tim-3 antibody.

FIG. 11 shows the same experiment as FIG. 10, with PD-1 antibodies used instead of anti-Tim-3 antibodies.

FIG. 12 shows the same experiment as FIG. 10 or 11, with CTLA-4 antibodies used.

FIG. 13 shows the effect of IL-2 complex treatment on reduction of exhausted CD8⁺ T cells, as measured by PD-1 levels on CD8⁺ T cells. Mice were injected with B16F10 melanoma cells, followed by treatment with phosphate-buffered saline (PBS, red curve with peak at 3×10E3 cells) or IL-2 complexes (IL-2-Cx, blue curve with peak at 4×10E2 cells) for 4 days (namely, d4, d5, d6, and d7). Analysis of PD-1 expression on different CD8⁺ T cell subsets (namely, activated memory-phenotype CD44⁺ CD122⁻ [filled black], resting memory-phenotype CD44⁺ CD122⁺[dotted], and naive CD44⁻ CD122⁻ [blank]) within tumour-infiltrating lymphocytes (TILs) and tumour-draining lymph nodes (TDLNs) was performed by flow cytometry on day 16 after tumour inoculation. Shown are histograms of PD-1 expression on CD8⁺ T cells from TILs of mice receiving PBS (red line) or IL-2-Cx (blue line) (A), as well as mean fluorescence intensity (MFI) values of PD-1 expression in the indicated CD8⁺ T cell subsets from either TILs (B) or TDLNs (C).

FIG. 14 shows the effect of IL-2 complex treatment on tumour infiltrating lymphocytes (TIL): Mice were injected with B16F10 melanoma cells, treatment with IL-2 complexes was performed for 4 days (namely, d4, d5, d6, and d7) and analysis of spleen cells (A) and TILs (B) was performed at day 16.
A) upper left: x-axis: <Pacific Blue-A>: CD3, y-axis: <APC-Cy-A>: CD8, event count: 178587; upper right: x-axis: <FITC-A>: CD122, y-axis: <PerCP-Cy5-5-A>: CD44, event count: 20590; lower left: x-axis: <Pacific Blue-A>: CD3, y-axis: <APC-Cy-A>: CD8, event count: 73331; lower right: x-axis: <FITC-A>: CD122, y-axis: <PerCP-Cy5-5-A>: CD44, event count: 20840.
B) upper left: x-axis: <Pacific Blue-A>: CD3, y-axis: <APC-Cy-A>: CD8, event count: 10837; upper right: x-axis: <FITC-A>: CD122, y-axis: <PerCP-Cy5-5-A>: CD44, event count: 2943; lower left: x-axis: <Pacific Blue-A>: CD3, y axis: <APC-Cy-A>: CD8, event count: 17749; lower right: x-axis: <FITC-A>: CD122, y-axis: <PerCP-Cy5-5-A>: CD44, event count: 5856.

### ITEMS

1. A combination medicament comprising
   a. a human interleukin-2 (hIL-2)-specific monoclonal antibody (mAb), or antigen binding fragment thereof, wherein binding of said antibody to hIL-2 inhibits binding of hIL-2 to CD25, and the antibody is characterized by any one of the parameters:
      i. the variable chain of the mAb comprises an amino acid sequence having an identity of ≥85%, ≥90%, ≥95%, or ≥99% compared to SEQ ID NO 005 and/or SEQ ID NO 006;
      ii. the binding to hIL-2 is characterized by a dissociation constant (K_{D}) ≤7.5 nmol/L, ≤5 nmol/L, ≤3 nmol/L, ≤2 nmol/L or ≤1.5 nmol/L;
      iii. the binding to hIL-2 is characterized by an off-rate (K_{off})≤1×10⁻⁴ s⁻¹, ≤8×10⁻⁵ s⁻¹, ≤6×10⁻⁵ s⁻¹, ≤4×10⁻⁵ s⁻¹, ≤3×10⁻⁵ s⁻¹ or ≤2.1×10⁻⁵ s⁻¹;
      iv. the antibody or antigen binding fragment thereof binds to a specific human interleukin-2 (hIL-2) epitope which comprises the amino acids K52, P54, K55, T57, R58, T61, F62, K63, Q94, and K96 of hIL-2; and/or
      v. the antibody displays no measurable cross-reactivity to murine IL-2;
   b. an immune checkpoint inhibitor agent selected from
      i. an inhibitor of interaction of CTLA-4 with CD80 or CD86,
      ii. an inhibitor of PD-1/PD-L1 interaction, and
      iii. a ligand of TIM-3
      iv. an inhibitor of the interaction of BTLA with HVEM
      v. an inhibitor of the interaction of LAG3 with galectin 3.
2. The combination medicament according to item 1, wherein the human interleukin-2 (hIL-2) specific monoclonal antibody, or antigen binding fragment thereof comprises at least one V_{H} and/or one V_{L} sequence having an identity of ≥80%, ≥85%, ≥90%, ≥92%, ≥93%, ≥94%, ≥95%, ≥96%, ≥97% or ≥98% compared to SEQ ID NOs 019 and/or 20.
3. The combination medicament according to any one of the preceding items, characterized in the human interleukin-2 (hIL-2) specific monoclonal antibody, or antigen binding fragment thereof, comprises at least one complementarity determining region (CDR) sequence having an identity of ≥80%, ≥85%, ≥90%, ≥92%, ≥93%, ≥94%, ≥95%, ≥96%, ≥97% or ≥98% compared to SEQ ID NOs 007, 008, 009, 010, 011 or 012, particularly wherein said human interleukin-2 (hIL-2) specific monoclonal antibody, or antigen binding fragment thereof, comprises three, four, or even more particularly five or six different CDR sequences, wherein each of said CDR sequences is selected from SEQ ID NOs 007, 008, 009, 010, 011 or 012 or from a sequence ≥90%, ≥92%, ≥93%, ≥94%, ≥95%, ≥96%, ≥97% or ≥98% or 100% identical thereto.
4. The combination medicament according to any one of the preceding items, wherein the human interleukin-2 (hIL-2) specific monoclonal antibody, or antigen binding fragment thereof is encoded by a nucleic acid molecule that has ≥60%, ≥70%, ≥80%, ≥85%, ≥90%, ≥92%, ≥93%, ≥94%, ≥95%, ≥96%, ≥97% or 98% sequence identity compared to SEQ ID NOs 003 and/or 004.
5. The combination medicament according to any one of the preceding items, wherein the human interleukin-2 (hIL-2) specific monoclonal antibody, or antigen binding fragment thereof is encoded by a nucleic acid molecule comprising a sequence tract having the sequence of SEQ ID NOs 013, 014, 015, 016, 017, 018, 021 or 022, or a sequence having an identity of ≥80%, ≥85%, ≥90%, ≥92%, ≥93%, ≥94%, ≥95%, ≥96%, ≥97% or ≥98% compared thereto,
   particularly wherein said antibody or fragment thereof is encoded by a nucleic acid molecule comprising 3, 4, 5 or six sequence tracts each having a different sequence selected from SEQ ID NO 13, 14, 15, 16, 17 and 18 or a sequence ≥90%, ≥92%, ≥93%, ≥94%, ≥95%, ≥96%, ≥97% or ≥98% identical thereto.
6. The combination medicament according to any one of the preceding items, wherein the hIL-2 mAb or antigen binding fragment thereof binds to a human interleukin-2 (hIL-2) epitope that further comprises any one or more of the amino acids N50, N53, N91, L92, A93, and N97 of hIL-2.
7. The combination medicament according to any one of items 1 to 6, wherein the combination medicament further comprises recombinant human interleukin-2, either in wild-type form or containing amino acid mutations.
8. A combination medicament comprising
   a. a fusion protein comprising:
      i. a human interleukin-2 (hIL-2) specific binding polypeptide fragment, wherein said polypeptide fragment is characterized by any one of the parameters:
         binding of said polypeptide fragment to hIL-2 inhibits binding of hIL-2 to CD25; and/or
         the hIL-2 binding polypeptide fragment comprises an amino acid sequence having an identity of ≥85%, ≥90%, ≥92%, ≥93%, ≥94%, ≥95%, ≥96%, ≥97% or ≥98% compared to SEQ ID NO 019 and/or SEQ ID NO 020; and/or
         the binding to hIL-2 is characterized by a dissociation constant (K_{D}) ≤7.5 nmol/L, ≤5 nmol/L, ≤3 nmol/L, ≤2 nmol/L or ≤1.5 nmol/L; and/or
         the binding to hIL-2 is characterized by an off-rate (K_{off})^{≤}1×10⁻⁴ s⁻¹, ≤8×10⁻⁵ s⁻¹, ≤6×10⁻⁵ s⁻¹, ≤4×10⁻⁵ s⁻¹, ≤3×10⁻⁵ s⁻¹ or ≤2.1×10⁻⁵ s⁻¹; and/or
         the antibody or antigen binding fragment thereof binds to a specific human interleukin-2 (hIL-2) epitope which comprises the amino acids K52, P54, K55, T57, R58, T61, F62, K63, Q94, and K96 of hIL-2; and/or
         the antibody displays no measurable cross-reactivity to murine IL-2;
      ii. a human IL-2 polypeptide fragment having an identity of ≥85%, ≥90%, ≥92%, ≥93%, ≥94%, ≥95%, ≥96%, ≥97% or ≥98% compared to SEQ ID NO 001, and, optionally,
      iii. an amino acid linker of 1 to 50, particularly of 5 to 40, more particularly of 10 to 30, even more particularly of approx. 15 to 25 amino acids, linking the hIL-2 binding polypeptide fragment to the human IL-2 polypeptide fragment as one single polypeptide chain; and
   b. an immune checkpoint inhibitor selected from an inhibitor of CTLA-4 interaction with CD80 or CD86, an inhibitor of PD-1/PD-L1 interaction, a ligand of TIM-3, an inhibitor of the interaction of BTLA with HVEM, and an inhibitor of the interaction of LAG3 with galectin 3.
9. The combination medicament according to any one of the previous items, wherein the human interleukin-2 (hIL-2) specific binding polypeptide fragment binds to a human interleukin-2 (hIL-2) epitope that further comprises any one or more of the amino acids N50, N53, N91, L92, A93, and N97 of hIL-2.
10. The combination medicament according to any one of the previous items, wherein the immune checkpoint inhibitor agent is an antibody specifically binding to CTLA-4, CD80, CD86, PD-1, PD-L1, TIM-3, BTLA, HVEM, LAG3 or galectin 3.
11. The combination medicament according to item 11, wherein the antibody specifically binding to CTLA-4, CD80, CD86, PD-1, PD-L1, TIM-3, BTLA, HVEM, LAG3 or galectin 3 is a non-agonistic antibody.
12. The combination medicament according to any one of the previous items, wherein the immune checkpoint inhibitor agent is an inhibitor of interaction of CTLA-4 with CD80 or CD86.
13. The combination medicament according to any one of the previous items, wherein the immune checkpoint inhibitor agent is selected from the group of nivolumab (Opdivo; CAS No. 946414-94-4), pembrolizumab (Keytruda; CAS No. 1374853-91-4), atezolizumab (Tecentriq; CAS No. 1380723-44-3), or ipilimumab (Yervoy; CAS No. 477202-00-9).
14. The combination medicament according to any one of the previous items for use in therapy of cancer.
15. The combination medicament according to any one of the previous items for use in therapy of renal cell cancer, lung cancer, bladder cancer or malignant melanoma, particularly metastatic malignant melanoma.

### EXAMPLES

Human Interleukin-2 Specific Antibody

Until now, no monoclonal antibodies (mAbs) suitable for the disclosed invention have been available. The anti-human IL-2 mAbs disclosed herein allow crucial steps towards the use and commercialization of this technology in clinical applications:
Further sequencing and fine characterization of the anti-human IL-2 mAbs.
Humanization of the anti-human IL-2 mAbs, which is essential to avoid (or minimize) immunogenicity in patients.
Generation of different formats of anti-human IL-2 mAbs, such as IgG, IgG1, IgG4, Fab, and single-chain Fv (scFv), as well as other formats, including diabodies and minibodies.
Generation of a fusion protein consisting of human IL-2 and an anti-human IL-2 mAb (or a fragment of the anti-human IL-2 mAb): such a construct has the advantage of consisting of one component only, instead of two as in IL-2 bound to an anti-human IL-2 mAb.

The inventors have generated and characterized specific anti-human IL-2 mAbs that are able to bind human IL-2 and, when tested in mice, are able to exert specific and potent stimulation of cytotoxic lymphocytes, including CD8⁺ T cells and natural killer (NK) cells.

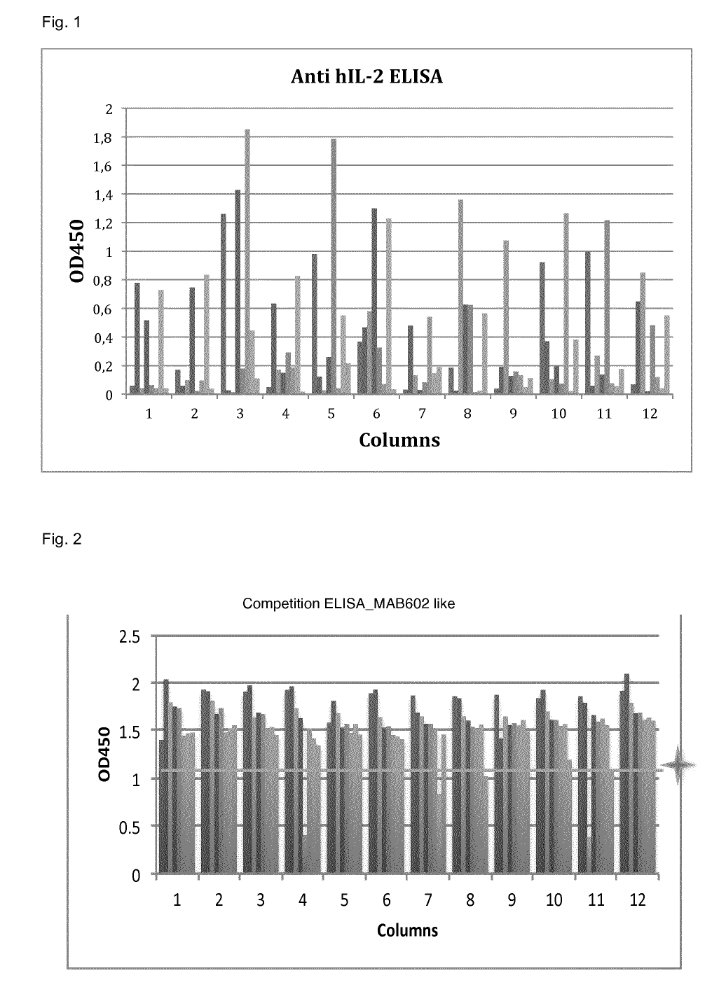

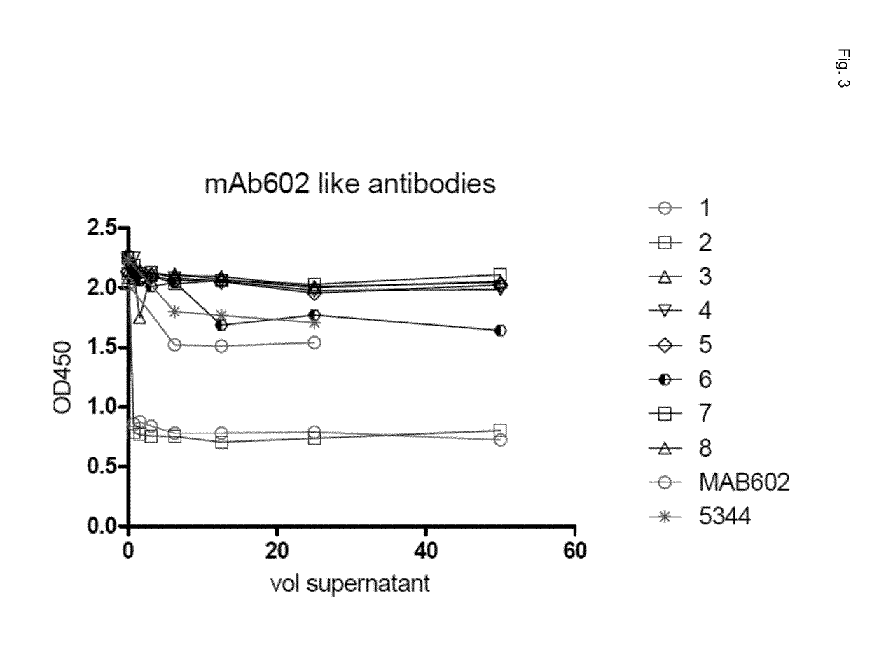

The inventors have developed specific screening assays that allow detection of specific anti-human IL-2 antibodies (so-called “binders”) in the serum of immunized animals and in the supernatant of the B cell clones obtained after B cell hybridoma fusion. In a second step it was discriminated between standard binders and those targeting a presumed specific epitope of the human IL-2 molecule. One example of such an in vitro enzyme-linked immunosorbent assay (ELISA) performed with different B cell clones, is shown in FIGS. 1 to 3.

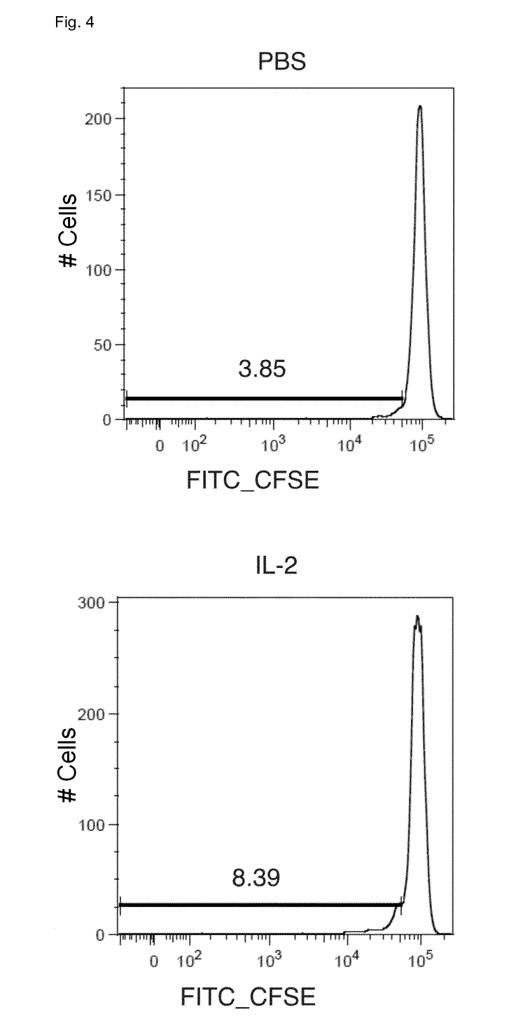

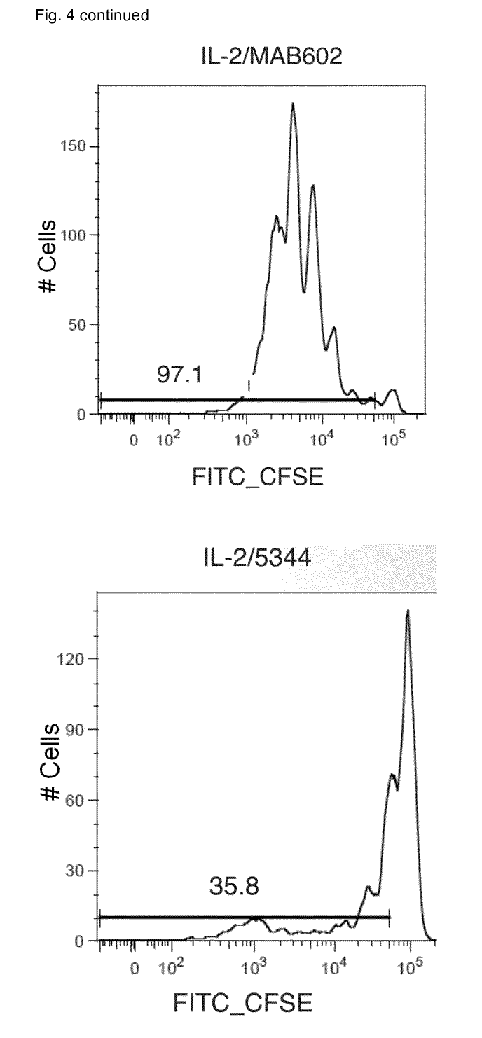

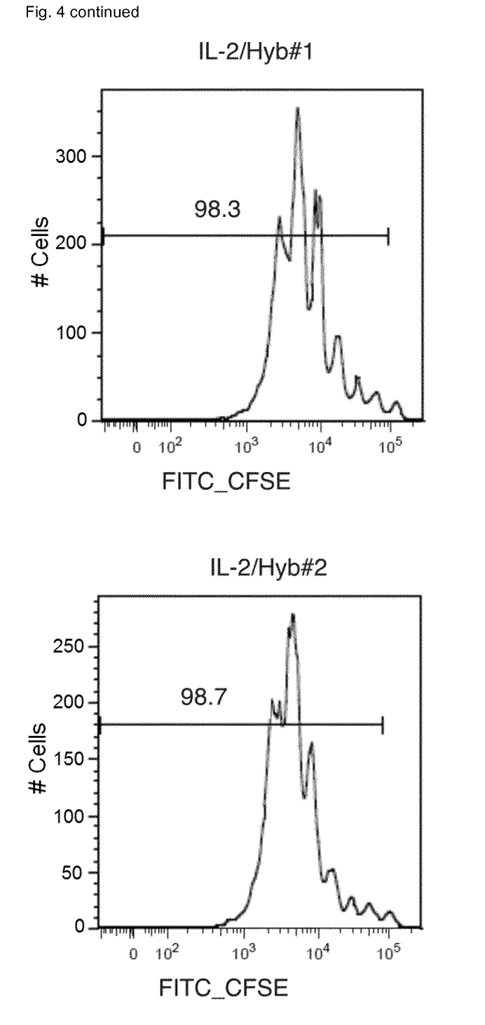

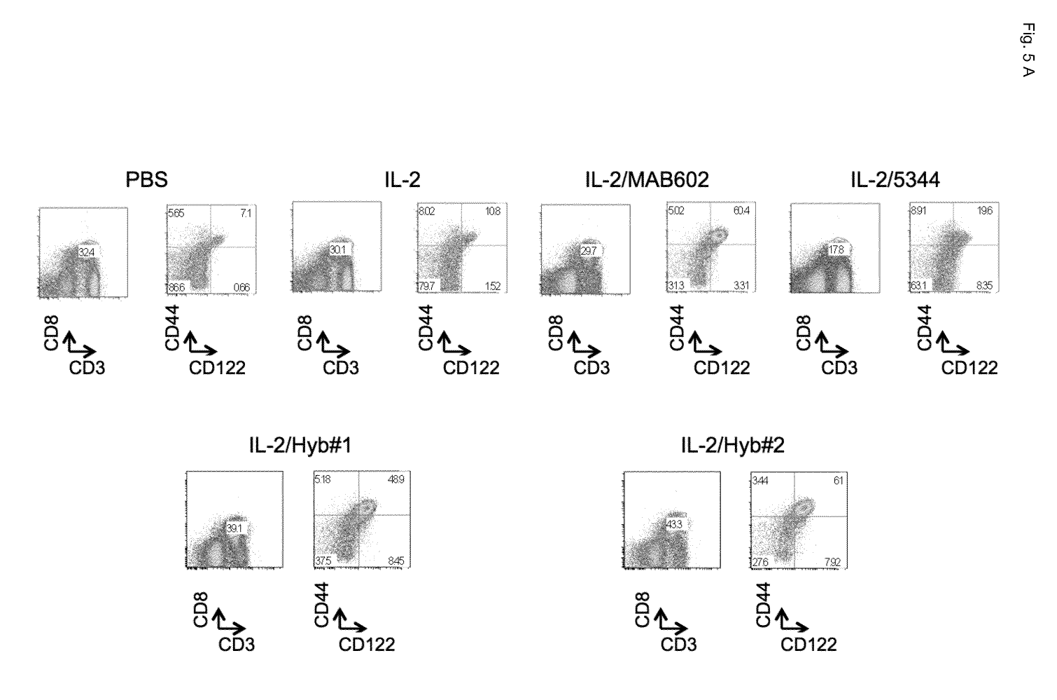

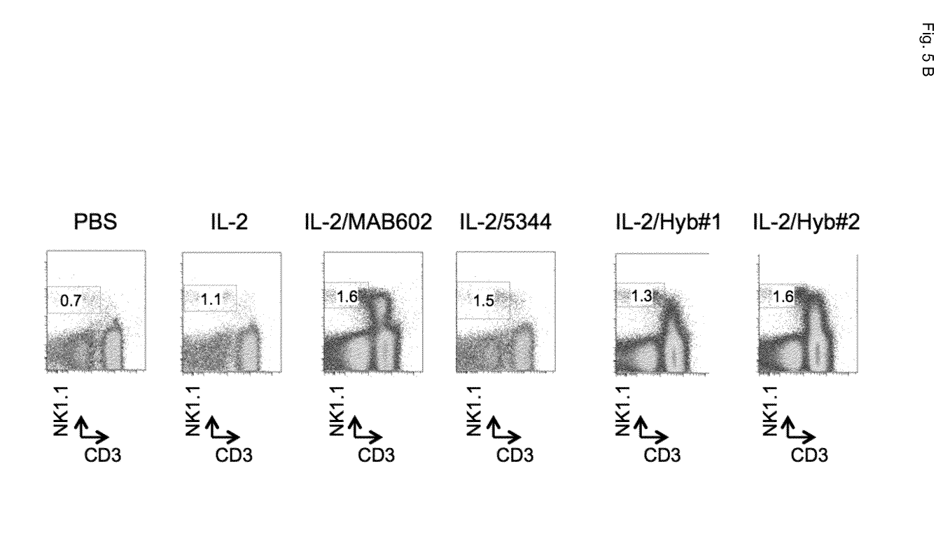

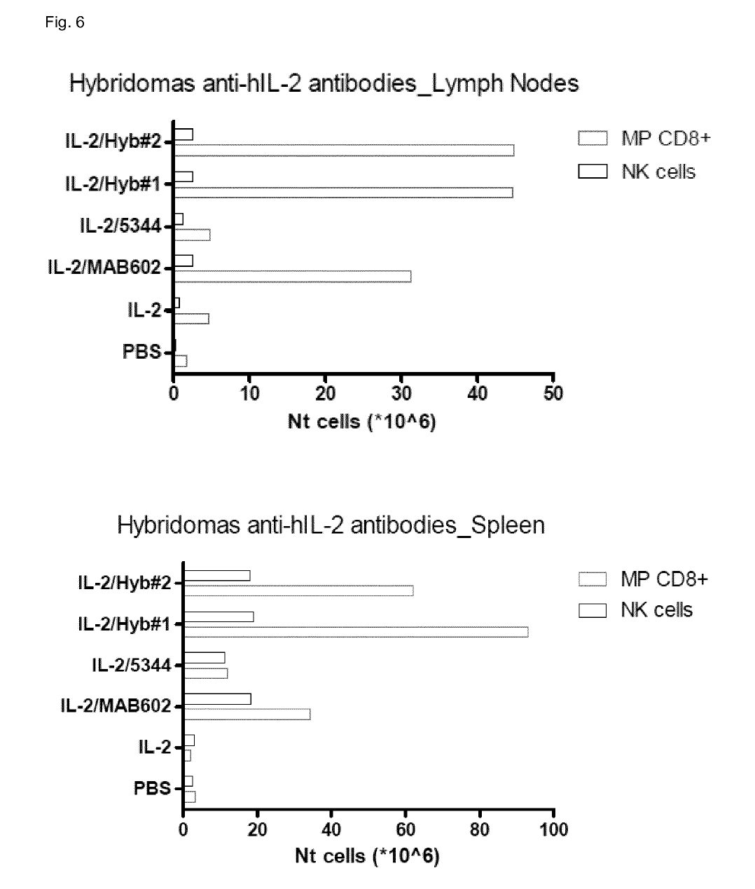

After the in vitro screening of the anti-human IL-2 mAbs, these mAbs were characterised in vivo. To this end and in order to obtain sufficient amounts of mAbs, the mAbs were concentrated from the supernatant of the hybridomas, the amount was estimated using an ELISA and finally the anti-human IL-2 mAbs was tested in mice. The results obtained on proliferation and expansion of CD8⁺ T cells and NK cells is shown in FIGS. 4 to 6.

In order to characterize the binding properties of the anti-human IL-2 mAbs the binding to human interleukin-2 was tested with surface plasmon resonance binding assays. The commercially available anti-human IL-2 mAb MAB602 was measured as a comparison. In FIG. 7 binding curves of MAB602 (left graph) and NARA1 (an antibody according to this invention; right graph) to human interleukin-2 at varying concentrations are shown. The dissociation constant (K_{D}) as well as the rate constants Kₒₙ and K_{off} measured for MAB602 and NARA1 are shown in table 1.

TABLE 1
Binding properties of anti-human IL-2 mAbs to human IL-2

| | Kₒₙ (M*s⁻¹) | K_{off} (s⁻¹) | K_{D} (nM) |
|---|---|---|---|
| MAB602 | 5.8 × 10⁴ | 4.94 × 10⁻⁴ | 9.7 |
| NARA1 | 1.78 × 10⁴ | 2.08 × 10⁻⁵ | 1.2 |

(Human interleukin-2 protein; P60568; 153aa):
SEQ ID NO 001
MYRMQLLSCIALSLALVTNSAPTSSSTKKTQLQLEHLLLDLQMILNGINNYKNPKLTRMLTFKFYMPK
KATELKHLQCLEEELKPLEEVLNLAQSKNFHLRPRDLISNINVIVLELKGSETTFMCEYADETATIVE
FLNRWITFCQSIISTLT
(Proleukin):
SEQ ID NO 002
MAPTSSSTKKTQLQLEHLLLDLQMILNGINNYKNPKLTRMLTFKFYMPKKATELKHLQCLEEELKPLE
EVLNLAQSKNFHLRPRDLISNINVIVLELKGSETTFMCEYADETATIVEFLNRWITFSQSIISTLT
(Heavy chain DNA sequence; 1413 bp):
SEQ ID NO 003
ATGGAATGGAGCGGAGTCTTTATCTTTCTCCTGTCAGTAACTGCAGGTGTTCACTCCCAGGTCCAGCT
GCAGCAGTCTGGAGCTGAGCTGGTAAGGCCTGGGACTTCAGTGAAGGTGTCCTGCAAGGCTTCTGGAT
ACGCCTTCACTAATTACTTGATAGAGTGGGTAAAGCAGAGGCCTGGACAGGGCCTTGAGTGGATTGGA
GTGATTAATCCTGGAAGTGGTGGTACTAACTACAATGAGAAGTTCAAGGGCAAGGCAACACTGACTGC
AGACAAATCCTCCAGCACTGCCTACATGCAGCTCAGCAGCCTGACATCTGATGACTCTGCGGTCTATT
TCTGTGCAAGATGGAGGGGGGATGGTTACTACGCGTACTTCGATGTCTGGGGCGCAGGGACCACGGTC
ACCGTCTCCTCAGCCAAAACAACAGCCCCATCGGTCTATCCACTGGCCCCTGTGTGTGGAGATACAAC
TGGCTCCTCGGTGACTCTAGGATGCCTGGTCAAGGGTTATTTCCCTGAGCCAGTGACCTTGACCTGGA
ACTCTGGATCCCTGTCCAGTGGTGTGCACACCTTCCCAGCTGTCCTGCAGTCTGACCTCTACACCCTC
AGCAGCTCAGTGACTGTAACCTCGAGCACCTGGCCCAGCCAGTCCATCACCTGCAATGTGGCCCACCC
GGCAAGCAGCACCAAGGTGGACAAGAAAATTGAGCCCAGAGGGCCCACAATCAAGCCCTGTCCTCCAT
GCAAATGCCCAGCACCTAACCTCTTGGGTGGACCATCCGTCTTCATCTTCCCTCCAAAGATCAAGGAT
GTACTCATGATCTCCCTGAGCCCCATAGTCACATGTGTGGTGGTGGATGTGAGCGAGGATGACCCAGA
TGTCCAGATCAGCTGGTTTGTGAACAACGTGGAAGTACACACAGCTCAGACACAAACCCATAGAGAGG
ATTACAACAGTACTCTCCGGGTGGTCAGTGCCCTCCCCATCCAGCACCAGGACTGGATGAGTGGCAAG
GAGTTCAAATGCAAGGTCAACAACAAAGACCTCCCAGCGCCCATCGAGAGAACCATCTCAAAACCCAA
AGGGTCAGTAAGAGCTCCACAGGTATATGTCTTGCCTCCACCAGAAGAAGAGATGACTAAGAAACAGG
TCACTCTGACCTGCATGGTCACAGACTTCATGCCTGAAGACATTTACGTGGAGTGGACCAACAACGGG
AAAACAGAGCTAAACTACAAGAACACTGAACCAGTCCTGGACTCTGATGGTTCTTACTTCATGTACAG
CAAGCTGAGAGTGGAAAAGAAGAACTGGGTGGAAAGAAATAGCTACTCCTGTTCAGTGGTCCACGAGG
GTCTGCACAATCACCACACGACTAAGAGCTTCTCCCGGACTCCGGGTAAATGA
(Light chain DNA sequence; 717 bp):
SEQ ID NO 004
ATGGAGACAGACACAATCCTGCTATGGGTGCTGCTGCTCTGGGTTCCAGGCTCCACTGGTGACATTGT
GCTGACCCAATCTCCAGCTTCTTTGGCTGTGTCTCTAGGGCAGAGGGCCACCATCTCCTGCAAGGCCA
GCCAAAGTGTTGATTATGATGGTGATAGTTATATGAACTGGTACCAACAGAAACCAGGACAGCCACCC
AAACTCCTCATCTATGCTGCATCCAATCTAGAATCTGGGATCCCAGCCAGGTTTAGTGGCAGTGGGTC
TGGGACAGACTTCACCCTCAACATCCATCCTGTGGAGGAGGAGGATGCTGCAACCTATTACTGTCAGC
AAAGTAATGAGGATCCGTACACGTTCGGAGGGGGGACCAAGCTGGAAATAAAACGGGCTGATGCTGCA
CCAACTGTATCCATCTTCCCACCATCCAGTGAGCAGTTAACATCTGGAGGTGCCTCAGTCGTGTGCTT
CTTGAACAACTTCTACCCCAAAGACATCAATGTCAAGTGGAAGATTGATGGCAGTGAACGACAAAATG
GCGTCCTGAACAGTTGGACTGATCAGGACAGCAAAGACAGCACCTACAGCATGAGCAGCACCCTCACG
TTGACCAAGGACGAGTATGAACGACATAACAGCTATACCTGTGAGGCCACTCACAAGACATCAACTTC
ACCCATTGTCAAGAGCTTCAACAGGAATGAGTGTTAG
(Heavy chain amino acid sequence; 470aa):
SEQ ID NO 005
MEWSGVFIFLLSVTAGVHSQVQLQQSGAELVRPGTSVKVSCKASGYAFTNYLIEWVKQRPGQGLEWIG
VINPGSGGTNYNEKFKGKATLTADKSSSTAYMQLSSLTSDDSAVYFCARWRGDGYYAYFDVWGAGTTV
TVSSAKTTAPSVYPLAPVCGDTTGSSVTLGCLVKGYFPEPVTLTWNSGSLSSGVHTFPAVLQSDLYTL
SSSVTVTSSTWPSQSITCNVAHPASSTKVDKKIEPRGPTIKPCPPCKCPAPNLLGGPSVFIFPPKIKD
VLMISLSPIVTCVVVDVSEDDPDVQISWFVNNVEVHTAQTQTHREDYNSTLRVVSALPIQHQDWMSGK
EFKCKVNNKDLPAPIERTISKPKGSVRAPQVYVLPPPEEEMTKKQVTLTCMVTDFMPEDIYVEWTNNG
KTELNYKNTEPVLDSDGSYFMYSKLRVEKKNWVERNSYSCSVVHEGLHNHHTTKSFSRTPGK
(Light chain amino acid sequence; 238aa):
SEQ ID NO 006
METDTILLWVLLLWVPGSTGDIVLTQSPASLAVSLGQRATISCKASQSVDYDGDSYMNWYQQKPGQPP
KLLIYAASNLESGIPARFSGSGSGTDFTLNIHPVEEEDAATYYCQQSNEDPYTFGGGTKLEIKRADAA
PTVSIFPPSSEQLTSGGASVVCFLNNFYPKDINVKWKIDGSERQNGVLNSWTDQDSKDSTYSMSSTLT
LTKDEYERHNSYTCEATHKTSTSPIVKSFNRNEC
(Heavy chain CDR1 amino acid sequence; 5aa):
SEQ ID NO 007
NYLIE
(Heavy chain CDR2 amino acid sequence; 17aa):
SEQ ID NO 008
VINPGSGGTNYNEKFKG
(Heavy chain CDR3 amino acid sequence; 12aa):
SEQ ID NO 009
WRGDGYYAYFDV
(Light chain CDR1 amino acid sequence; 15aa):
SEQ ID NO 010
KASQSVDYDGDSYMN
(Light chain CDR2 amino acid sequence; 7aa):
SEQ ID NO 011
AASNLES
(Light chain CDR3 amino acid sequence; 9aa):
SEQ ID NO 012
QQSNEDPYT
(Heavy chain CDR1 DNA sequence; 15bp):
SEQ ID NO 013
AATTACTTGATAGAG
(Heavy chain CDR2 DNA sequence; 51bp):
SEQ ID NO 014
GTGATTAATCCTGGAAGTGGTGGTACTAACTACAATGAGAAGTTCAAGGGC
(Heavy chain CDR3 DNA sequence; 36bp):
SEQ ID NO 015
TGGAGGGGGGATGGTTACTACGCGTACTTCGATGTC
(Light chain CDR1 DNA sequence; 45bp):
SEQ ID NO 016
AAGGCCAGCCAAAGTGTTGATTATGATGGTGATAGTTATATGAAC
(Light chain CDR2 DNA sequence; 21bp):
SEQ ID NO 017
GCTGCATCCAATCTAGAATCT
(Light chain CDR3 DNA sequence; 21bp):
SEQ ID NO 018

CAGCAAAGTAATGAGGATCCGTACACG
(Heavy chain variable region (V_{H}), amino acid sequence;
121aa):

SEQ ID NO 019
QVQLQQSGAELVRPGTSVKVSCKASGYAFTNYLIEWVKQRPGQGLEWIGVINPGSGGTNYNEKFKGKA
TLTADKSSSTAYMQLSSLTSDDSAVYFCARWRGDGYYAYFDVWGAGTTVTVSS
(Light chain variable region (V_{L}), amino acid sequence;
111aa):
SEQ ID NO 020
DIVLTQSPASLAVSLGQRATISCKASQSVDYDGDSYMNWYQQKPGQPPKLLIYAASNLESGIPARFSG
SGSGTDFTLNIHPVEEEDAATYYCQQSNEDPYTFGGGTKLEIK
(Heavy chain variable region (V_{H}), DNA sequence;
363bp):
SEQ ID NO 021
CAGGTCCAGCTGCAGCAGTCTGGAGCTGAGCTGGTAAGGCCTGGGACTTCAGTGAAGGTGTCCTGCAA
GGCTTCTGGATACGCCTTCACTAATTACTTGATAGAGTGGGTAAAGCAGAGGCCTGGACAGGGCCTTG
AGTGGATTGGAGTGATTAATCCTGGAAGTGGTGGTACTAACTACAATGAGAAGTTCAAGGGCAAGGCA
ACACTGACTGCAGACAAATCCTCCAGCACTGCCTACATGCAGCTCAGCAGCCTGACATCTGATGACTC
TGCGGTCTATTTCTGTGCAAGATGGAGGGGGGATGGTTACTACGCGTACTTCGATGTCTGGGGCGCAG
GGACCACGGTCACCGTCTCCTCA
(Light chain variable region (V_{L}), DNA sequence;
333bp):
SEQ ID NO 022
GACATTGTGCTGACCCAATCTCCAGCTTCTTTGGCTGTGTCTCTAGGGCAGAGGGCCACCATCTCCTG
CAAGGCCAGCCAAAGTGTTGATTATGATGGTGATAGTTATATGAACTGGTACCAACAGAAACCAGGAC
AGCCACCCAAACTCCTCATCTATGCTGCATCCAATCTAGAATCTGGGATCCCAGCCAGGTTTAGTGGC
AGTGGGTCTGGGACAGACTTCACCCTCAACATCCATCCTGTGGAGGAGGAGGATGCTGCAACCTATTA
CTGTCAGCAAAGTAATGAGGATCCGTACACGTTCGGAGGGGGGACCAAGCTGGAAATAAAA
(IL-2-(GxS)y-heavy chain; C-terminus of hIL-2 fused to
N-terminus of variable heavy chain of NARA1):
SEQ ID NO 023
MYRMQLLSCIALSLALVTNSA P T S S S T K K T Q L Q L E H L L L D L Q M I
L N G I N N Y K N P K L T R M L T F K F Y M P K K A T E L K H L Q C
L E E E L K P L E E V L N L A Q S K N F H L R P R D L I S N I N V I
V L E L K G S E T T F M C E Y A D E T A T I V E F L N R W I T F C Q
S I I S T L T G G G G S G G G G S G G G G S G G
QVQLQQSGAELVRPGTSVKVSCKASGYAFTNYLIEWVKQRPGQGLEWIGVINPGSGGTNYNEKFKGKA
TLTADKSSSTAYMQLSSLTSDDSAVYFCARWRGDGYYAYFDVWGAGTTVTVSSAKTTAPSVYPLAPVC
GDTTGSSVTLGCLVKGYFPEPVTLTWNSGSLSSGVHTFPAVLQSDLYTLSSSVTVTSSTWPSQSITCN
VAHPASSTKVDKKIEPRGPTIKPCPPCKCPAPNLLGGPSVFIFPPKIKDVLMISLSPIVTCVVVDVSE
DDPDVQISWFVNNVEVHTAQTQTHREDYNSTLRVVSALPIQHQDWMSGKEFKCKVNNKDLPAPIERTI
SKPKGSVRAPQVYVLPPPEEEMTKKQVTLTCMVTDFMPEDIYVEWTNNGKTELNYKNTEPVLDSDGSY
FMYSKLRVEKKNWVERNSYSCSVVHEGLHNHHTTKSFSRTPG
(IL-2-(GxS)y-light chain; C-terminus of hIL-2 fused to
N-terminus of variable light chain of NARA1):
SEQ ID NO 024
MYRMQLLSCIALSLALVTNSA P T S S S T K K T Q L Q L E H L L L D L Q M I
L N G I N N Y K N P K L T R M L T F K F Y M P K K A T E L K H L Q C
L E E E L K P L E E V L N L A Q S K N F H L R P R D L I S N I N V I
V L E L K G S E T T F M C E Y A D E T A T I V E F L N R W I T F C Q
S I I S T L T G G G G S G G G G S G G G G S G G
DIVLTQSPASLAVSLGQRATISCKASQSVDYDGDSYMNWYQQKPGQPPKLLIYAASNLESGIPARFSG
SGSGTDFTLNIHPVEEEDAATYYCQQSNEDPYTFGGGTKLEIKRADAAPTVSIFPPSSEQLTSGGASV
VCFLNNFYPKDINVKWKIDGSERQNGVLNSWTDQDSKDSTYSMSSTLTLTKDEYERHNSYTCEATHKT
STSPIVKSFNRNEC

(underlined sequence tracts in SEQ ID NO 23, 24 correspond to IL-2 sequence part).

Combination Medicament

Mice

3-month-old female C57Bl/6J mice were purchased (Charles River Laboratories). No statistical methods were used to predetermine sample size. For all experiments presented in this study, the sample size was large enough to measure the effect size. The experiments were not randomized and the investigators were not blinded to allocation during experiments and outcome assessment. Mouse colonies were maintained in certified animal facilities in accordance with Swiss guidelines. All animal experiments were approved by the veterinary authorities of Canton of Zurich, Switzerland, and were performed in accordance with Swiss law and the GlaxoSmithKline policy on the Care, Welfare, and Treatment of Animals. Pre-established exclusion criteria were based on the Canton of Zurich veterinary authority's guidelines and included substantial weight loss of >15% of initial body weight. During the study period most of the animals appeared to be in good health.

Cell Cultures

The murine B16-F10 melanoma cell line was purchased (ATCC). Cells were cultured in growth medium, which was RPMI 1640 (42401, Life Technologies) supplemented with 10% FCS (16140, Life Technologies), 4 mM L-Glutamine (25030, Life Technologies), Penicillin-Streptomycin (15070, Life Technologies), and Fungizone Antimycotic (15290, Life Technologies).

Grafting of Murine Melanoma Cells

Recipient mice were intradermally engrafted with 1×10⁶ B16-F10 cells. Mice engrafted with melanoma cells were sacrificed not later than at a time point defined by tumor volume (V >1′000 mm³). Tumor volume was calculated as follows: V=2/3×π×((a+b)/4)³, a (mm) was the length and b (mm) was the width of the tumor.

In Vivo Treatments

Recombinant human IL-2 (IL-2, Teceleukin, Roche), anti-CTLA-4 mAb (9D9, BioXcell), anti-TIM-3 mAb (RMT3-23, BioXcell), anti-PD-1 mAb (RMP1-14, BioXcell) and GSK503 (GlaxoSmithKline) were purchased. IL-2cx were prepared by mixing IL-2 (1.5 μg corresponding to 15′000 IU) and anti-IL-2 mAb (1 μg), as previously described [Letourneau, S., et al., Proc Natl Acad Sci USA, 2010. 107(5): p. 2171-6]. Treatment of B16-F10-engrafted mice was started, when tumors became visible and palpable (days 3-5) [Krieg, C., et al., Proc Natl Acad Sci USA, 2010. 107(26): p. 11906-11]. Where indicated, mice received intraperitoneal injections of IL-2cx, 250 μg of anti-CTLA-4 mAb, or 250 μg of anti-PD-1 mAb or 250 μg of anti-TIM-3 antibody.

Flow Cytometry

Single cell suspensions of spleen and lymph nodes were prepared according to standard protocols. Tumors were cut into small pieces, pooled per groups in order to obtain enough cells for analysis, and incubated in 10 ml dissociation buffer (RPMI, 5% FCS, 10 μg/ml DNAase I [D4527, Sigma-Aldrich] and 200 U/ml collagenase type I [17100-017, Life Technologies]) for 45 minutes at 37° C. and 25 rpm. Cell suspensions were then passed through a 70 μm cell strainer. After one wash a Percoll (17-5445-01, GE Healthcare) gradient centrifugation was performed. All cell suspensions were stained for flow cytometry analysis using PBS with 2% FCS, 2 mM EDTA and fluorochrome-conjugated Abs (Table 2). Samples were acquired with a FACS Canto and analyzed using FlowJo Software.

TABLE 2
Antibodies used for flow cytometry.

| Antigen | Fluorophore | Company | Serial number |
|---|---|---|---|
| CD122 | FITC | eBioscience | 12-2281 |
| CD3 | PB | BD Biosciences | 558214 |
| CD44 | PerCP-Cyanine5.5 | eBioscience | 45-0441 |
| CD8a | PerCP-eFluor 710 | eBioscience | 46-0081 |
| PD-1 | APC | eBioscience | 17-9985 |
| CD45.2 | PE | eBioscience | 12-0454 |

1. A combination medicament comprising
a. a human interleukin-2 (hIL-2)-specific monoclonal antibody (mAb), or antigen binding fragment thereof, wherein binding of said antibody to hIL-2 inhibits binding of hIL-2 to CD25, and the antibody is characterized by any one of the parameters:
i. the variable chain of the mAb comprises an amino acid sequence having an identity of ≥85%, ≥90%, ≥95%, or ≥99% compared to SEQ ID NO 005 or SEQ ID NO 006;
ii. the binding to hIL-2 is characterized by a dissociation constant (K_{D}) ≤7.5 nmol/L, ≤5 nmol/L, ≤3 nmol/L, ≤2 nmol/L or ≤1.5 nmol/L;
iii. the binding to hIL-2 is characterized by an off-rate (K_{off})≤1×10⁴ s⁻¹, ≤8×10⁵ s⁻¹, ≤6×10⁵ s⁻¹, ≤4×10⁵ s⁻¹, ≤3×10⁻⁵ s⁻¹ or ≤2.1×10⁻⁵ s⁻¹;
iv. the antibody or antigen binding fragment thereof binds to a specific human interleukin-2 (hIL-2) epitope which comprises the amino acids K52, P54, K55, T57, R58, T61, F62, K63, Q94, and K96 of hIL-2; and/or
v. the antibody displays no measurable cross-reactivity to murine IL-2.
b. an immune checkpoint inhibitor agent selected from
i. an inhibitor of interaction of CTLA-4 with CD80 or CD86,
ii. an inhibitor of PD-1/PD-L1 interaction, and
iii. a ligand of TIM-3.

2. The combination medicament according to claim 1, wherein said human interleukin-2 (hIL-2) specific monoclonal antibody, or antigen binding fragment thereof comprises at least one V_{H} and/or one V_{L} sequence having an identity of ≥80%, ≥85%, ≥90%, ≥92%, ≥93%, ≥94%, ≥95%, ≥96%, ≥97% or ≥98% compared to SEQ ID NOs 019 or 20.

3. The combination medicament according to claim 1, characterized in that said human interleukin-2 (hIL-2) specific monoclonal antibody, or antigen binding fragment thereof, comprises at least one complementarity determining (CDR) sequence having an identity of ≥80%, ≥85%, ≥90%, ≥92%, ≥93%, ≥94%, ≥95%, ≥96%, ≥97% or ≥98% compared to SEQ ID NOs 007, 008, 009, 010, 011 or 012.

4. The combination medicament according to claim 1, wherein said human interleukin-2 (hIL-2) specific monoclonal antibody, or antigen binding fragment thereof is encoded by a nucleic acid molecule that has ≥60%, ≥70%, ≥80%, ≥85%, ≥90%, ≥92%, ≥93%, ≥94%, ≥95%, ≥96%, ≥97% or ≥98% sequence identity compared to SEQ ID NOs 003 or 004.

5. The combination medicament according to claim 1, wherein said human interleukin-2 (hIL-2) specific monoclonal antibody, or antigen binding fragment thereof is encoded by a nucleic acid molecule having the sequence of SEQ ID NOs 13, 14, 15, 16, 17, 18, 21 or 22, or a sequence having an identity of ≥80%, ≥85%, ≥90%, ≥92%, ≥93%, ≥94%, ≥95%, ≥96%, ≥97% or ≥98% compared thereto.

6. The combination medicament according to claim 1, wherein the hIL-2 mAb or antigen binding fragment thereof binds to a human interleukin-2 (hIL-2) epitope that further comprises any one or more of the amino acids N50, N53, N91, L92, A93, and N97.

7. The combination medicament according to claim 1, wherein the combination medicament further comprises recombinant human interleukin-2, either in wild-type form or containing amino acid mutations.

8. A combination medicament comprising
a. a fusion protein comprising:
i. a human interleukin-2 (hIL-2) specific binding polypeptide fragment, wherein said polypeptide fragment is characterized by any one of the parameters:
binding of said polypeptide fragment to hIL-2 inhibits binding of hIL-2 to CD25, and/or
the hIL-2 binding polypeptide fragment comprises an amino acid sequence having an identity of ≥85%, ≥90%, ≥92%, ≥93%, ≥94%, ≥95%, ≥96%, ≥97% or ≥98% compared to SEQ ID NO 021 or SEQ ID NO 022, and/or
the binding to hIL-2 is characterized by a dissociation constant (K_{D}) ≤7.5 nmol/L, ≤5 nmol/L, ≤3 nmol/L, ≤2 nmol/L or ≤1.5 nmol/L; and/or
the binding to hIL-2 is characterized by an off-rate (K_{off}) ≤1×10⁴ s⁻¹, ≤8×10⁻⁵ s⁻¹, ≤6×10⁻⁵ s⁻¹, ≤4×10⁻⁵ s⁻¹, ≤3×10⁻⁵ s⁻¹ or ≤2.1×10⁻⁵ s⁻¹; and/or
the antibody or antigen binding fragment thereof binds to a specific human interleukin-2 (hIL-2) epitope which comprises the amino acids K52, P54, K55, T57, R58, T61, F62, K63, Q94, and K96 of hIL-2;
the antibody displays no measurable cross-reactivity to murine IL-2;
ii. a human IL-2 polypeptide fragment having an identity of ≥85%, ≥90%, ≥92%, ≥93%, ≥94%, ≥95%, ≥96%, ≥97% or ≥98% compared to SEQ ID NO 001, and, optionally,
iii. an amino acid linker of 1 to 50, particularly of 5 to 40, more particularly of 10 to 30, even more particularly of approx. 15 to 25 amino acids, linking the hIL-2 binding polypeptide fragment to the human IL-2 polypeptide fragment as one single polypeptide chain;
and
b. an immune checkpoint inhibitor selected from an inhibitor of CTLA-4 interaction with CD80 or CD86, an inhibitor of PD-1/PDL-1 interaction, and a ligand of TIM-3.

9. The combination medicament according to claim 1, wherein the human interleukin-2 (hIL-2) specific binding polypeptide fragment binds to a human interleukin-2 (hIL-2) epitope that further comprises any one or more of the amino acids N50, N53, N91, L92, A93, and N97.

10. The combination medicament according to claim 1, wherein said immune checkpoint inhibitor agent is an inhibitor of interaction of CTLA-4 with CD80 or CD86.

11. The combination medicament according to claim 1, wherein said immune checkpoint inhibitor agent is ipilimumab (Yervoy; CAS No. 477202-00-9).

12. The combination medicament according to claim 1 the previous claims for use in therapy of cancer.

13. The combination medicament according to claim 1 for use in therapy of malignant melanoma, particularly metastatic malignant melanoma.
